(19) United States
(12) Patent Application Publication
(10) Pub. No.: US 2023/0183582 A1
(43) Pub. Date: Jun. 15, 2023

(54) PROCESS FOR CONVERTING SOLID HYDROCARBONACEOUS MATERIALS TO CHEMICALS AND FUELS

(71) Applicant: Anellotech, Inc., Pearl River, NY (US)

(72) Inventors: Omar M. Basha, Chadds Ford, PA (US); Leslaw Mleczko, Dormagen (DE); Terry J. Mazanec, Solon, OH (US); Harley Freeman, Pearl River, NY (US)

(21) Appl. No.: 17/549,898

(22) Filed: Dec. 14, 2021

(51) Int. Cl. C10G 11/18; C10G 1/00; C10G 1/10; B01J 29/40; C07C 4/06; B01J 8/18; B01J 8/24; B01D 45/16

(52) U.S. Cl. CPC C10G 11/182; C10G 1/002; C10G 11/185; C10G 1/10; B01J 29/40; C07C 4/06; B01J 8/1827; B01J 8/24; B01D 45/16; C10G 2300/1003; C10G 2400/20; C10G 2400/30; C10G 2300/706; C07C 2529/40

(57) ABSTRACT

Processes of catalytically pyrolyzing solid hydrocarbonaceous materials in a downflow fluid bed reactor and regenerating the catalyst in an upflow fluidized bed reactor are described. Systems and compositions useful in the catalytic pyrolysis of plastics are also described.

### FIELD OF THE INVENTION

This invention relates to the conversion of waste plastics, polymers, and other waste or biomass materials to useful chemical and fuel products such as paraffins, olefins, and aromatics in a catalytic pyrolysis process with catalyst regeneration in an upflow reactor.

### BACKGROUND

In 2018, plastics generation in the United States was 38.5 million tons, which was 13.1 percent of MSW generation. World-wide over 350 million tons of plastics were produced. Plastic recycling recovers scrap or waste plastic and reprocesses the material into useful products. However, since China banned the import of waste plastics, the recycle rate in the US is estimated to have dropped to only 4.4%. Plastic recycling is challenging because of the chemical nature of the long chain organic polymers and low economic returns. In addition, waste plastic materials often need sorting into the various plastic resin types, e.g. low density polyethylene (LDPE), high density polyethylene (HDPE), polypropylene (PP), polystyrene (PS), polyvinyl chloride (PVC), and polyethylene-terephthalate (PET) for separate recycling treatments.

Plas-TCat™ is a catalytic fluid bed process using zeolite catalysts for converting solid, hydrocarbonaceous feedstocks comprising polymer/plastic material, and optionally biomass, to a mixed product of permanent gases, C2-C4 light olefins, C1-C4 light paraffins, and C5+ hydrocarbons including benzene, toluene, and xylenes (“BTX”), aromatic and non-aromatic naphtha range molecules, C11+ hydrocarbons, coke and char, and minor byproducts. Conversion occurs in a fluid bed reactor using ZSM-5 zeolite or similar catalyst. A portion of the light gases produced by the reaction may be recycled to the reactor to provide fluidization gas and for feedstock injection into the vessel. Coke and char by-products that accumulate on the catalyst and temporarily deactivate it are removed by oxidation in a continuously operating catalyst regenerator. Waste materials which can be processed by Plas-TCat™ include biomass, waste tires, lubricating oils, coal, and petroleum residues in addition to plastics.

US Patent Application 2013/0172173 by Mukthiyar describes a process and apparatus for catalyst regeneration from more than one FCC unit to control carbon content on the catalyst.

Pradeep et al, in U.S. Pat. No. 9,434,892 describe a two-step process for catalytically cracking heavy liquid hydrocarbon feeds like vacuum gasoil, atmospheric tower bottoms, or vacuum residue, wherein fresh feed is reacted with a partially deactivated catalyst and partially processed feed is reacted with a more active catalyst to produce olefins and distillates.

In U.S. Pat. No. 10,130,929, to Indian Oil Corp., Sadhullah et al describe an apparatus for conducting FCC using a compact unit containing concentric catalytic and catalyst regenerator reactors.

It is an object of the present invention to provide for the conversion of solid hydrocarbonaceous materials, such as waste plastics, polymers, biomass, and other materials, to useful chemical and fuel products such as paraffins, olefins, and BTX, wherein the catalyst is regenerated in an upflow regenerator.

### SUMMARY OF THE INVENTION

In a first aspect, the invention provides a process for converting solid hydrocarbonaceous materials to useful products comprising: feeding solid hydrocarbonaceous materials and fluidization fluid into a fluidized bed catalytic pyrolysis reactor containing a solid conversion catalyst, reacting the hydrocarbonaceous materials to form vapor products and, optionally, solid residue, withdrawing and recovering the vapor products, withdrawing the used catalyst and solids from the catalytic reactor, feeding the withdrawn used catalyst and any solids at or near the bottom of an upflow fluidized bed catalyst regenerator, regenerating the catalyst in the regenerator; withdrawing the hot, regenerated catalyst from the top of the regenerator, passing at least a portion of the hot, regenerated catalyst into a fluidized bed catalytic pyrolysis reactor; typically, the fluidized bed catalytic pyrolysis reactor is the same catalytic pyrolysis reactor as the reactor from which the catalyst is withdrawn; however, the regenerator could be in a series of reactors such as a circle of reactors with intervening regenerators; likewise there could any number of either reactors and/or regenerators in a system.

The invention can be further characterized by one or any combination of the following features: wherein the vapor products are separated from entrained catalyst and solids in one or more cyclones; wherein heat from the catalyst regenerator is used to heat the solid feed materials or fluidization fluid or both; wherein an amount of regenerated catalyst is removed from the separated stream of regenerated catalyst and discarded; wherein regenerated catalyst is removed and discarded each day in an amount from 0.1 to 5.0, or from 0.5 to 4.0, or from 1.0 to 3.0, or at least 0.1, or at least 0.5, or at least 1, or no more than 5.0, or no more than 4.0 or no more than 3.0% by mass of the separated, regenerated catalyst; wherein fresh catalyst is added to the catalytic pyrolysis reactor with the regenerated catalyst or from a separate conduit or some combination of these; wherein fresh catalyst is added to the catalytic pyrolysis reactor in an amount of fresh catalyst that is from 0.1 to 5.0, or from 0.5 to 4.0, or from 1.0 to 3.0, or at least 0.1, or at least 0.5, or at least 1, or no more than 5.0, or no more than 4.0 or no more than 3.0% by mass of the regenerated catalyst each day; wherein the solid hydrocarbonaceous materials are selected from biomass, polyethylene (PE), polypropylene (PP), polyacetylene, polybutylene, polyolefins, polyethylene terephthalate (PET), polybutylene terephthalate, copolyesters, polyester, polycarbonate, polyurethanes, polyamides, polystyrene (PS), polyacetal, epoxies, polycyanurates, polyacrylics, polyurea, vinyl esters, polyacrylonitrile, polyvinyl alcohol, polyvinylchloride (PVC), poly(methyl methacrylate) (PMMA), polyvinyl acetate, nylon, copolymers such as: ethylene-propylene, EPDM, acrylonitrile-butadiene-styrene (ABS), nitrile rubber, natural and synthetic rubber, tires, styrene-butadiene, styrene-acrylonitrile, styrene-isoprene, styrene-maleic anhydride, ethylene-vinyl acetate, nylon 12/6/66, filled polymers, polymer composites, polymer composites comprising natural fibers, plastic alloys, other polymeric materials, whether obtained from polymer or plastic manufacturing processes as waste or discarded materials, post-consumer recycled polymer materials, materials separated from waste streams such as municipal solid waste, black liquor, wood waste, or other biologically produced materials, or a combination of these; wherein the solid hydrocarbonaceous material is selected from among polyethylene, polypropylene, and polystyrene, or mixtures thereof; wherein the solid hydrocarbonaceous materials comprise from 30 to 100, or from 40 to 80, or from 45 to 70, or at least 30, or at least 40, or at least 45, or less than 99, or less than 95, or less than 80 percent by mass of a combination of polyethylene (PE) (sum of low- and high-density polyethylene), polypropylene (PP), and polystyrene (PS; wherein the solid hydrocarbonaceous materials comprise from 1 to 30, or 2 to 20, or 3 to 10, or up to 30, or up to 20 or up to 10, or at least 0.1, or at least 1, or at least 2% by mass biomass; wherein the solid hydrocarbonaceous materials comprise from 0.1 to 20, or from 1 to 15, or from 3 to 10, or at least 0.1, or at least 1, or at least 3, or less than 20, or less than 15, or less than 10 percent by mass polyethylene terephthalate (PET); wherein the solid hydrocarbonaceous materials comprise from 0.1 to 20, or from 1 to 15, or from 3 to 10, or at least 0.1, or at least 1, or at least 3, or less than 20, or less than 15, or less than 10 percent by mass nylon; wherein from 5 to 5000, or from 5 to 2500, or from 100 to 1500, or at least 25, or at least 100 metric tons per day (mtpd) of solid hydrocarbonaceous materials are processed in the catalytic pyrolysis reactor; wherein the weight hourly space velocity is from 0.1 to 2.0, or from 0.2 to 1.0, or from 0.25 to 0.75, or at least 0.1, or at least 0.2, or at least 0.25, or less than 2.0, or less than 1.0, or less than 0.75 hr⁻¹; wherein the residence time of an average carbon atom within the catalytic pyrolysis reactor is from 0.5 to 180, or from 2 to 100, or from 4 to 80, or from 10 to 60, or from 30 to 60, or at least 5, or at least 10, or at least 30, or less than 180, or less than 100, or less than 80, or less than 60 seconds; wherein the catalytic pyrolysis reactor is a fluidized bed reactor that comprises a sparger or distributor, located at or near the bottom of the reactor, that serves to distribute the fluidization fluid; wherein the superficial inlet gas velocity of the fluidization fluid entering the reactor is from 0.05 to 1.0, or from 0.1 to 0.7, or from 0.2 to 0.5, or at least 0.1, or at least 0.2, or at least 0.3, or less than 1.0, or less than 0.7, or less than 0.5 meters per second; wherein the fluidization fluid is an inert gas, or a hydrocarbon gas, or a recycle stream separated from the products, or a combination thereof; wherein the catalytic pyrolysis reactor is from 0.1 to 15, or from 0.3 to 10, or from 3 to 7, or at least 3, or at least 5 meters in diameter; wherein the height/diameter ratio (H:D) of the catalytic pyrolysis reactor is from 0.5:1 to 10:1, or from 1:1 to 8:1, or from 1.5:1 to 5:1, or at least 1:1, or at least 1.5:1, or at least 2:1, or less than 15:1, or less than 10:1, or less than 5:1; wherein the catalytic pyrolysis reaction is conducted in a fluidized bed chosen from among a bubbling bed, or turbulent bed, or some combination of these; wherein the catalytic pyrolysis is conducted at an operating temperature in the range from 450 to 750, or from 500 to 650, or from 550 to 600, or at least 450, or at least 500 or at least 550, or no more than 750, or no more than 650° C.; wherein the pressure in the catalytic pyrolysis reactor is from 0.5 to 10, or 1.0 to 6, or 1.5 to 4, or at least 1, or at least 1.5 or at least 2, or less than 20, or less than 10, or less than 6 barg; wherein the mass flow rate of the hot, regenerated catalyst recycle stream is from 3 to 1700, or from 170 to 845, or at least 60, or at least 150 or at least 310, or less than 170, or less than 360 or less than 845 kg/s; wherein the mass flow rate of the hot regenerated catalyst into the catalytic pyrolysis reactor, when defined as the amount of catalyst added per unit time divided by the amount of catalyst in the bed, is from 0.025% per second to 5% per second, or from 0.1% per second to 3.5% per second, or at least 0.5% per second, or at least 1.5% per second, or at least 3% per second, or less than 7% per second; wherein the vapor products from the catalytic pyrolysis comprises from 1-70 wt %, or 5-65 wt %, or 10-60 wt %, or 20-50 wt %, or 30-45 wt %, or 40-65 wt %, or 50-70 wt %, or at least 20 wt %, or at least 30 wt %, or at least 40 wt %, or at least 50 wt %, or at least 60 wt % C2-C4 alkenes; wherein the conversion catalyst comprises a zeolite; wherein the catalyst comprises a zeolite material with average pore sizes of less than 10, less than 5, less than 2, less than 1, less than 0.65, or between 0.5 and 10 nanometers; wherein the catalyst comprises a zeolite material with average pore sizes of between 0.55 and 0.65, or between 0.59 and 0.63 nanometers; wherein the catalyst comprises a zeolite material that has a constraint index (CI) value within the range of 1 to 12; wherein the catalyst is chosen from among ferrierite, zeolite Y, zeolite beta, mordenite, MCM-22, ZSM-5, ZSM-23, ZSM-57, SUZ-4, EU-1, ZSM-11, (S)A1PO-31, SSZ-23, or a combination thereof; wherein the catalyst comprises ZSM-5; wherein the catalyst comprises a metal, a metal oxide, or both, wherein the metal is chosen from among nickel, palladium, platinum, titanium, vanadium, chromium, manganese, iron, cobalt, zinc, copper, gallium, the rare earth elements, i.e., elements 57-71, cerium, zirconium, or a combination thereof; wherein a stream enriched in ethylene or propylene, or both, is separated from the condensable higher materials in the vapor products; wherein the vapor products from the catalytic pyrolysis comprise from 1-70 wt %, or 5-65 wt %, or 10-60 wt %, or 20-50 wt %, or 30-45 wt %, or 40-65 wt %, or 50-70 wt %, or at least 20 wt %, or at least 30 wt %, or at least 40 wt %, or at least 50 wt %, or at least 60 wt % C2-C4 alkenes; wherein an olefin-containing product stream is separated from the condensable higher materials in the vapor products that comprises at least 20, or at least 50, or at least 70, or in the range of 20 to 95, or 50 to 90, or 70 to 90 mass % olefins, or more; wherein the mass yield of olefins is at least 1%, or at least 2.5%, or at least 5%, or at least 8%, or at least 9%, or no more than 40%, or no more than 25%, or no more than 15%, or from 1% to 40%, or from 3% to 28%, or from 5% to 15%, and the mass yield of all products is no more than 100% based on the mass in the feed; wherein a stream comprising C5+ products is separated from the vapor products; wherein a stream comprising benzene, toluene, xylenes, or some combination of these (BTX) is separated from the vapor products; wherein the mass yield of BTX is at least 16%, or at least 22%, or at least 30%, or at least 40%, or at least 50%, or at least 55%, or at least 60%, or from 15% to 75%, or from 20% to 70%, or from 45% to 65%, and the mass yield of all products is no more than 100% based on the mass of solid hydrocarbonaceous materials fed to the process; wherein the mass yield of olefins plus aromatics is greater than 40%, or greater than 60% or greater than 70%, or greater than 75%, or greater than 80%, or from 40% to 99%, or from 60% to 95%, or from 65% to 90%, and the mass yield of all products is no more than 100% based on the mass of solid hydrocarbonaceous materials fed to the process; wherein the selectivity of ethylene as a percentage of the total olefins produced is at least 20%, or at least 25%, or at least 30%, or from 10% to 60%, or from 20% to 45%, or from 25% to 35%, and the selectivity of propylene as a percentage of the total olefins produced is at least 20%, or at least 30%, or at least 40%, or at least 45%, or at least 50%, or from 20% to 70%, or from 25% to 65%, or from 28% to 55%, such that the total selectivity of ethylene plus propylene is less than 100; wherein the selectivity of benzene plus toluene plus xylenes (BTX) as a percentage of aromatics produced is at least 40%, or at least 50%, or at least 80%, or at least 90%, or at least 95%, or at least 97%, or from 40% to 99.9%, or from 50% to 99.5%, or from 80 to 99%, or from 95% to 98; wherein the mass yield of coke and char from the catalytic pyrolysis is less than 10%, or less than 5%, or less than 2%, or less than 1%, or less than 0.5%, or from 0.1% to 10%, or from 0.2% to 5%, or from 0.3 to 2%, and the mass yield of all products is no more than 100% based on the mass of solid hydrocarbonaceous materials fed to the process; wherein the vapor products mixture is subjected to a separation process to produce a separated stream of gases comprising gases chosen from among any of C1 to C4 hydrocarbons, H2, CO₂ and CO, or some combination thereof; and passing at least a portion of the separated stream of gases to the regenerator as part of the fluidization fluid; wherein olefins are separated from the catalytic pyrolysis products for upgrading to BTX or other valuable products, and at least a portion of the olefins are recycled to the catalytic pyrolysis reactor; wherein olefins are separated from the catalytic pyrolysis products for upgrading to BTX or other valuable products, and the products of the upgrading are combined with products of the catalytic process for separation and purification; wherein an internal diameter of the catalytic pyrolysis reactor increases along at least a portion of the height of the reactor; wherein the internal diameter of the catalytic pyrolysis reactor increases along at least a portion of the height of the reactor, the portion being shaped like a cone where the angle of the wall of the cone with respect to vertical can range from 3 degrees to 50 degrees, or from 5 to 40, or from 7 to 25, or from 8 to 15, or at least 3, or at least 7, or at least 8 degrees, or at least 10 degrees from vertical, where vertical is defined as zero degrees; wherein the catalytic pyrolysis reactor comprises a conical portion of the reactor that includes at least the height of the dense bubbling phase of the fluid bed; wherein the catalytic pyrolysis reactor comprises a conical portion and wherein any additional height of the reactor above the top of the dense bubbling phase of the fluid bed is cylindrical; wherein the catalytic pyrolysis reactor comprises a conical portion and wherein the ratio of the superficial velocity of the vapors at the top of the conical portion of the reactor is no more than 1.01:1, or no more than 1.5:1, or no more than 2.0:1, or no more than 2.5:1, or no more than 3.0:1, or from 1.01:1 to 3.0:1, or from 1.1:1 to 2.0:1 compared to the superficial velocity of the vapors at the bottom of the conical portion of the reactor; wherein an oxidizing agent is fed to the regenerator via a gas feed stream; wherein the regenerator fluidization fluid is oxygen, air, recycled flue gas, or steam, or some combination of these; wherein the regenerator temperature is at least 300, or 400, or 500, or 600, or 700, or 800, or from 500 to 1000, or from 600 to 800, or from 650 to 700° C.; wherein the residence time of the catalyst in the regenerator is from 25 to 500, or from 50 to 300, or from 75 to 150, or at least 25, or at least 50, or at least 75, or at least 100, or no more than 500, or no more than 300 or no more than 150 seconds; wherein the solids flux, which is the rate of mass flow of solid material through a cross sectional area of the regenerator, is in the range from 19 to 300, or 50 to 250, or 100 to 200, or at least 19 or at least 50, or at least 100, or at least 150, or no more than 300, or no more than 250, or no more than 200 kg/m²s; wherein the superficial fluidization gas velocity at the entrance of the regenerator is at least 1.5, or at least 2.0, or at least 2.5, or at least 3.0, or from 0.6 to 7.5, or from 1.0 to 6.0, or from 1.5 to 5.0, or from 2.5 to 4.5, or from 3.0 to 4.0, or no more than 7.5, or no more than 6.5, or no more than 5.0, or no more than 4.0 m/s; wherein the height to diameter ratio (H/D) of the regenerator is from 2 to 15, or from 3 to 22, or from 5 to 40, or from 10 to 44, or at least 2, or at least 5, or at least 10, or no more than 10, or no more than 15, or no more than 30, or no more than 44; wherein at least a portion of the gases in the vapor product mixture is combusted in the regenerator.

The methods, systems and apparatus of the invention can also be conducted in conjunction with a separate thermal pyrolysis process before the catalytic conversion process. Thus, the invention can be further characterized by one or any combination of the following features: wherein a feed mixture comprising plastics is pyrolyzed in a thermal pyrolysis reactor and at least a portion of the products is fed to the catalytic pyrolysis reactor as a vapor; wherein the thermal pyrolysis reactor comprises a fluid bed reactor, or extruder, or rotating kiln reactor, or other suitable reactor that produces a vapor stream and a solids stream; wherein the thermal pyrolysis reactor comprises a fluid bed thermal pyrolysis reactor comprising heat transfer materials; wherein the heat transfer materials in the thermal pyrolysis reactor comprise an inert fluidizable material; wherein the heat transfer materials may be separated in one or more cyclones, regenerated in a regenerator, and a portion of the solids returned to the thermal pyrolysis reactor; wherein the fluidization fluid of the thermal pyrolysis reactor is an inert gas, or a hydrocarbon gas, or a recycle stream separated from the products, or a combination thereof; wherein the thermal pyrolysis reactor comprises an extruder, rotating kiln, or other reactor, the materials that are not converted to vapors are separated in the thermal pyrolysis step, and the separated materials are combusted to generate heat or energy or are discarded; wherein the thermal pyrolysis reactor is operated at temperatures from 400 to 700, or from 450 to 650, or from 500 to 600, or at least 400, or at least 450, or at least 500, or no more than 700, or no more than 650, or no more than 600° C.; wherein at least a portion of the heat required for the thermal pyrolysis is provided by shear heating of the polymer from mechanical work or a mechanical/moving/rotating device, such as an extruder or other device, that induces shear heating within the molten plastic mixture; wherein the residence time of the solid feed materials in the thermal pyrolysis reactor is from 1 to 30, or from 2 to 20, or from 5 to 15, or at least 1, or at least 2, or at least 5, or no more than 30, or no more than 20 or no more than 15 minutes; wherein the pressure in the thermal pyrolysis reactor can be from 1 to 30, from 2 to 20, from 3 to 10, or at least 2, or at least 3 or at least 4, or no more than 30, or no more than 20 or no more than 10 barg.

In addition, or separately, the invention provides a process of catalytically pyrolyzing a mixed feed of materials, comprising: feeding solid hydrocarbonaceous materials and fluidization fluid comprising 0.6 vol % to 10 vol % oxygen into a fluidized bed catalytic pyrolysis reactor containing a conversion catalyst, reacting the hydrocarbonaceous materials to form vapor products and, optionally, solid residue, withdrawing and recovering the vapor products, withdrawing catalyst and solids from the catalytic reactor, feeding the spent catalyst and any solids at or near the bottom of an upflow fluidized bed catalyst regenerator, withdrawing the hot, regenerated catalyst from the top of the regenerator, separating the hot, regenerated catalyst from the combustion vapors in one or more cyclones, passing at least a portion of the hot, regenerated catalyst into the catalytic reactor.

The invention can be further characterized by one or any combination of the following features: wherein an amount of oxygen is introduced into the feed stream that is from 0.6% to 8%, 1% to 6%, or from 2% to 4% by weight, or at least 0.5%, at least 2%, at least 4%, or at least 6% by mass of the mass of the feed stream; wherein the oxygen is introduced by the addition of air or O₂ preferably as a component of the fluidization fluid, or with the gas injected with the plastics, or by separate, direct injection into the fluidized bed, or a combination thereof; wherein an amount of oxygen is introduced into the process that is at least enough such that combustion of feed materials or other components with the introduced oxygen increases the temperature of the reacting mixture in the catalytic pyrolysis reactor by at least 25° C., or at least 100° C., or at least 200° C., or at least 300° C., or from 100° C. to 400° C.; wherein the oxygen is introduced by the addition of air or O₂ preferably as a component of the fluidization fluid, or with the gas injected with the plastics, or by separate, direct injection into the fluidized bed, or a combination thereof; wherein at least a portion of a stream comprising ethane and propane, or C4 olefins and paraffins, or a combination of them, that has been separated from the reaction products, is recycled to the catalytic pyrolysis reactor as a component of the fluidization gas.

In another aspect, the invention provides an apparatus for converting solid hydrocarbonaceous materials to useful products comprising: a feed hopper comprising solid hydrocarbonaceous materials comprising at least 50 mass percent polymers and optionally biomass, a fluidized bed catalytic pyrolysis reactor containing a conversion catalyst, a screw auger or pneumatic conveyor adapted to feed solid hydrocarbonaceous materials into the fluidized bed catalytic pyrolysis reactor, a conduit adapted to feed regenerated catalyst at a point in the reactor above the height of a dense bubbling phase of a fluid bed, a conduit adapted to withdraw catalyst and solids from the catalytic reactor, a conduit adapted to feed used catalyst and any solids withdrawn from the catalytic reactor at or near the bottom of an upflow fluidized bed catalyst regenerator, a conduit adapted to withdraw the hot, regenerated catalyst from the top of the regenerator, and one or more cyclones through which the hot regenerated catalyst and combustion products can be passed to separate the solids.

Likewise, the invention provides a system in which the various steps are actually occurring. The invention can be further characterized by one or any combination of the following features: wherein the conversion catalyst used in the catalytic pyrolysis comprises a zeolite; wherein the conversion catalyst comprises a zeolite that has a constraint index in the range 1 to 12; wherein the conversion catalyst comprises ZSM-5; wherein a heating zone is provided wherein at least a portion of the solid hydrocarbonaceous materials is heated to provide a molten mass that is hot filtered to remove suspended solids or impurities; wherein the step of pyrolyzing comprises fast solid pyrolysis in the presence of a catalyst; wherein the catalytic pyrolysis reactor is a fluidized bed or bubbling bed reactor at an operating temperature in the range from 450 to 750, or from 500 to 650, or from 550 to 600, or at least 450, or at least 500 or at least 550, or no more than 750, or no more than 650° C.; wherein the feed mixture for the catalytic pyrolysis process comprises, 10 to 95 or at least 10, 20, 30, 50, 60, or 10 to 80 mass % polymers; wherein the feed mixture comprises polyethylene, polypropylene, polystyrene, or some combination thereof; wherein the feed mixture of comprises biomass, cellulose, cotton clothing, PET, PET clothing, nylon, cellulose acetate, or some combination of these; wherein the screw auger or pneumatic conveyor feeding solid hydrocarbonaceous materials feeds a thermal pyrolysis reactor to convert the feed to a vapor stream and a solids stream, and wherein the vapor stream is fed to the catalytic pyrolysis reactor.

### BRIEF DESCRIPTION OF THE DRAWINGS

FIG. 1 illustrates a process for converting plastic-containing materials to fuels and chemicals using an upflow catalyst regenerator.

FIG. 2 illustrates a process that includes a pyrolysis step before the catalytic pyrolysis.

FIG. 3 schematically illustrates a drop tube reactor schematic.

FIG. 4 shows calculated olefins yield as a function of temperature. The higher space velocity are the lower curves.

FIG. 5 shows calculated paraffins yield as a function of temperature. The higher space velocity are the upper curves.

FIG. 6 shows calculated aromatics yield as a function of temperature. The higher space velocity are the upper curves.

FIG. 7 shows calculated local superficial gas velocity as a function of distance from the distributor for cylindrical and conical reactors.

### DISCUSSION

Transport of the catalyst within a fluidized bed process can be adjusted to improve process performance and reduce costs. Catalyst transport is operated under a variety of constraints. Used catalyst must be moved from the catalytic reactor to the regenerator, and the regenerated catalyst must be moved back to a catalytic reactor, while allowing for removal of a portion of used catalyst and addition of fresh catalyst as needed. Thus, a combination of a catalyst upflow in one process and downflow in the other process, is used in conventional fluid bed catalytic processes.

For plastics that are available as particulate solids, several seconds of time at reactor temperature are required to permit melting of the material before it can sufficiently mix with the catalyst and the thermal pyrolysis products can react with the catalyst. Operating the catalytic reactor as an upflow transport reactor cannot be conducted because the residence times are less than 5 seconds in risers of reasonable heights (<20 meters), and operating at velocities greater than 4 m/s as required for the riser will prohibit feeding of the plastic particles from the top since their minimum fluidization velocity of <1 m/s will cause them to ‘float’ on top of the column, minimizing mixing with the catalyst. Feeding polymer particles from the bottom of an upflow reactor is unsuitable since as the melting polymer coats the catalyst the particles will collide, agglomerate, and grow such that the system suffers ‘defluidization’, as described in “Defluidization Phenomena During the Pyrolysis of Two Plastic Wastes,” Arena, U., and M. L. Mastellone, Chem. Eng. Sci., 55, 2849, 2000. To achieve fluidization velocities in an upflow riser catalytic reactor requires a large volume of lift gases, that must be heated to process conditions, requiring considerable energy and cost. Similarly, operating an upflow reactor at lower superficial gas velocities, will require a lift line or jet to transport and introduce the plastic and catalyst into the reactor for sufficient mixing, which will also increase the risk of defluidization as mentioned earlier. Pre-mixing catalyst with polymer to feed the process is problematic because it requires handling and metering a highly viscous molten slurry that increases the risk of plugging; adding a mixing vessel does not overcome this problem because it is expensive and the fluidization velocity required for such premixed catalyst and polymer is much higher than for the catalyst alone, also adding costs.

Moreover, operating a plastics catalytic pyrolysis process in an upflow configuration presents problems of ensuring contact of the initially formed pyrolysis product gases with the catalyst. As the material rises, bubbles form and condense into larger bubbles, separating from the molten phase, bypassing the catalyst, and exiting the reactor. The too-short contact time of initial pyrolysis products with the catalyst cannot be overcome merely by increasing the height of the reactor since it will exceed the conventional hydrodynamic limitation of H/D (height/diameter)=4, nor can it be avoided by installing bubble breakers or other internals, since the high viscosity of the molten plastics, increases the risk of defluidization.

Due to the nature of the thermal pyrolysis reaction, increased axial gas velocity as much as 2× to 5× occurs as polymers are broken into smaller fragments that occupy larger volumes as vapors. For the upflow catalytic reactor design that requires a high velocity lift gas stream to introduce the catalyst into the reactor, the added gas volume will lead to extremely high velocities in the upper portions of the riser reactor that will reduce residence times unless the riser is dramatically increased in height far beyond 20 or 30 meters, moreover it will increase the amount of catalyst being elutriated and will require higher capacity cyclones to separate them out, increasing costs.

Scaleup of an upflow reactor and downflow regenerator system with expected capacity from 25 to 2500 tons per day or higher is not feasible because ancillary equipment associated with heating the lift gas, plus mixing challenges and defluidization risks will significantly increase capital costs. Concurrently, a downflow regenerator operating as a bubbling bed will require a large catalyst inventory to maintain catalyst activity sufficient for the process. In the bubbling bed regenerator, the more active (regenerated) and less active (deactivated) catalyst portions are mixed as in a stirred tank so that catalyst being removed and sent to the reactor has an activity that is the average of the catalyst activity in the regenerator that spans from the deactivated catalyst to the fully regenerated catalyst, thus requiring large volumes of catalyst to obtain a more active catalyst for passage to the reactor.

Advantages of a Downflow Catalytic Pyrolysis Reactor and Upflow Catalyst Regenerator

The present invention that transports catalyst in a downflow direction in the catalytic reactor and in an upflow direction in the catalyst regenerator overcomes or mitigates these drawbacks of an upflow catalytic pyrolysis reactor, a downflow regenerator system, or both.

A bubbling bed, turbulent bed, or similar catalytic pyrolysis reactor as specified in this invention permits long residence times of the feed material in contact with the catalyst as well as top feed of the catalyst to the catalytic pyrolysis reactor which eases mixing the molten reaction feed mixture with the catalyst. Polymer cracking has been shown to require at least about 50-60 seconds at temperatures of 500° C. or higher, or about 15-20 seconds at temperatures of 550° C. or higher to achieve complete thermal pyrolysis. Catalyst can be withdrawn for regeneration from either a location near the bottom of the reactor (within the bottom 10% of the height) or at some higher point in the bubbling or turbulent bed but below the top of the reactor, thus establishing a net downflow of catalyst that provides better performance than an upflow reactor. Similarly, the feed materials, that are typically particles of polymer, can be readily fed either from above the bed, or through a dedicated feed injection port nearer the bottom of the bed, optionally entrained in a fluidization gas. Since the gas velocity required to maintain fluidization in a bubbling bed is less than for a riser, smaller volumes of fluidization gas are used, and less energy is required for heating these gases. In the present invention, product vapors exit the reactor above the bubbling or turbulent bed establishing a net counter current flow of catalyst and feed.

The bubbling or turbulent bed reactor of the present invention allows for better contact between feed and initially formed pyrolysis products and catalyst due to the lower velocity of the gases and smaller bubbles formed. In addition, a bubbling or turbulent bed can be adapted to include a portion of the bed to be of increasing diameter along its axis, forming a cone, that permits the flow velocity along the axis to remain nearly constant as gas molecules are evolved in the pyrolysis process. Such a conical geometry with a larger cross section at the top and smaller cross section at the bottom is not viable for an upflow catalytic pyrolysis reactor due to the high initial flow velocity of the fluidization gas required to fluidize the material in the lower portion of the riser. At such high velocities the lift gas will bypass a large portion of the bottom conical configuration—channeling through selected areas—and result in mixing dead zones where feed contact with the catalyst is minimized.

An upflow regenerator can be readily scaled to larger scales without the need for large volumes of catalyst since little back-mixing occurs in the upflow regenerator, so that there is a gradient of the extent of regeneration that has the least active catalyst at the bottom and the most active, regenerated catalyst at the top. The riser regenerator also requires less heat for the oxygen-containing gas used as the oxidant since only a small portion of the gas needs to be heated to initiate the combustion and further regenerator gas can be added without heating at some higher point in the riser where the highly exothermic combustion has already heated the mixture; staged air addition can be used to cool the combusting mixture and control the temperature of the catalyst so that it need not be cooled significantly before being returned to the catalytic pyrolysis reactor.

### DETAILED DESCRIPTION

A process for producing useful chemicals or fuels comprises a mixture of solid feed materials that may comprise waste plastics, polymers, biomass, or other materials, that is fed to a downflow catalytic fluid bed reactor along with hot regenerated catalyst, the separation of valuable products of the catalytic pyrolysis conversion process, the upgrading of catalytic pyrolysis conversion products, the combustion of char, or coke, or selected byproducts of the catalytic pyrolysis, or other materials, in an upflow fluidized bed catalyst regenerator, the return of hot, regenerated catalyst to the top of the catalytic reactor, or some combination of these.

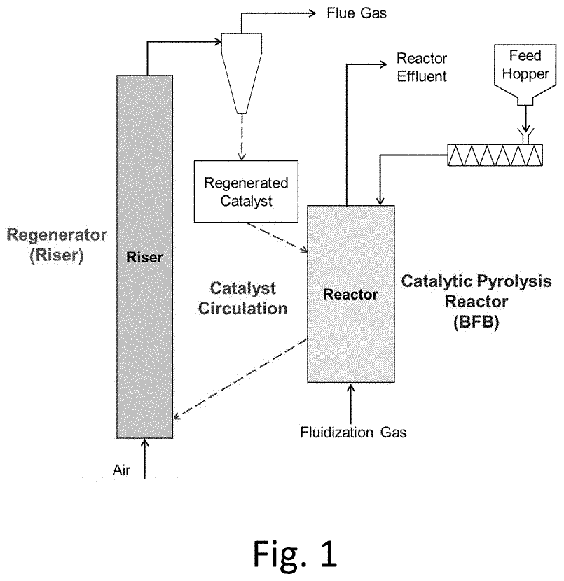

FIG. 1 presents a schematic of one embodiment of the process of the present invention in which solid hydrocarbonaceous Feed is introduced from a Feed Hopper via a screw auger or other mechanical or pneumatic means into a Catalytic Pyrolysis Reactor along with Regenerated Catalyst and Fluidization Gas, catalytic pyrolysis is conducted in the Reactor, the Reactor Effluent is separated and valuable products are recovered therefrom, catalyst is removed from the Reactor and sent to an upflow Riser Catalyst Regenerator along with an oxidant (shown in FIG. 1 as Air), catalyst is regenerated in the Regenerator to remove coke, char, and carbonaceous deposits on the catalyst, the hot, regenerated catalyst is separated from the flue gas in one or more Cyclones, and the hot, regenerated catalyst is returned to the downflow reactor.

A feed mixture of plastics and other hydrocarbonaceous materials is supplied to a fluidized bed catalytic pyrolysis reactor where it is reacted to form a vapor product stream and a solid catalyst containing stream. The feed mixture may be fed to the fluidized bed reactor from above (with respect to gravity) as shown in FIG. 1 Error! Reference source not found. or may be fed at another point of the catalytic reactor. The feed may be fed into the reactor using a screw auger or by jet injection with a carrier gas, or other means. The feed may be melted to form a molten liquid that is fed to the reactor or evaporated and fed to the reactor as fluidization gas.

Catalyst fed to the fluidized bed reactor comprises a portion of the Regenerated Catalyst and may comprise fresh catalyst. Typically, as the catalyst activity decays, a small portion of catalyst is replaced regularly with fresh catalyst, preferably at a rate of about 1-3% per day. Additional fresh catalyst may be added to the system via a makeup stream. Fresh catalyst may be added to the catalytic pyrolysis reactor with the regenerated catalyst, may be added separately, may be added with the feed materials, or any combination of these.

In the fluidized bed reactor the hydrocarbonaceous materials are pyrolyzed and catalytically converted to a vapor stream containing useful products including olefins, paraffins, and aromatics, and which carries along entrained solid catalyst and other solids. The vapor products are separated from the entrained solids in one or more cyclones. At least a portion of the solid particles can be returned to the catalytic reactor, a portion of the solids may be removed, and a portion of the solids may be fed to the catalyst regenerator. The vapor products can be sent to a separation and recovery system to recover useful products such as paraffins, olefins, aromatics, or other products.

A catalyst containing stream is removed from the catalytic reactor and passed to a catalyst regenerator in which it is contacted with an oxidizing gas such as air to regenerate the catalyst and produce energy from the combustion. Catalyst can be removed from the catalytic pyrolysis reactor via an overflow standpipe, or a drainpipe, or another apparatus, as is commonly practiced with fluid bed reactor systems. Catalyst may optionally be removed at, or near, the bottom of the reactor. In either case the catalyst moves in a net downflow direction through the catalytic reactor. Catalyst is added at a position on the reactor above (with respect to gravity) where the catalyst is removed.

The vapor product stream from the catalytic pyrolysis is separated into valuable product streams containing olefins and aromatics, and a byproduct stream that may contain methane, ethane, propane, butane, H₂, CO₂, and CO. Optionally, a portion of the byproduct gas stream can be passed to the regenerator to increase the heat generation therein or used in the fluidization of the catalytic reactor. Energy for use in the process, e.g., for heating feed materials or recycle or fluidization gases or for other purposes, may be recovered from the hot combustion gases (Flue Gas) produced in the regenerator by heat exchange in one or more heat exchangers, or by other means. A portion of the energy generated in the catalyst regenerator can be used as thermal energy in the catalytic pyrolysis reactor, or for products separation, or both, or the energy can be converted to electrical energy, or the generated energy can be used as thermal energy and electrical energy within the plant or exported. At least a portion of the hot regenerated catalyst is returned to the catalytic pyrolysis reactor. The hot regenerated catalyst supplies heat to drive the catalytic pyrolysis process.

In one set of embodiments, an oxidizing agent is fed to the riser regenerator via a gas feed stream shown as ‘Air’ in FIG. 1 Error! Reference source not found.. A solid mixture comprising partially deactivated catalyst may comprise residual carbon and/or coke as well as coke or char from the process, which may be removed via reaction with the oxidizing agent in the regenerator. The oxidizing agent may originate from any source including, for example, a source of oxygen, atmospheric air, or steam, among others. In the regenerator, the catalyst is re-activated by reacting the catalyst with the oxidizing agent and heat is generated.

In some embodiments a portion of the gaseous products from the catalytic pyrolysis process is fed to the catalyst regenerator to be combusted with the solid materials. In other embodiments natural gas or other light hydrocarbons or droplets of liquid hydrocarbonaceous materials or small particles of solid hydrocarbonaceous materials or some combination of these may be fed to the catalyst regenerator, which combustion provides additional heat to the regenerator. The regenerator in FIG. 1 comprises a Flue Gas vent stream which may include regeneration reaction products, residual oxidizing agent, residual hydrocarbons etc.

As shown in the illustrative embodiment of FIG. 1, the regenerated catalyst may exit the regenerator, be separated from the vapors in one or more cyclones, and at least a portion of the catalyst is recycled back to the catalytic pyrolysis reactor via a recycle stream. The net movement of catalyst through the regenerator is in the upflow direction. In some cases, catalyst may be lost from the system during operation and replaced by fresh catalyst. In some cases, additional fresh catalyst may be added to the system via a makeup stream.

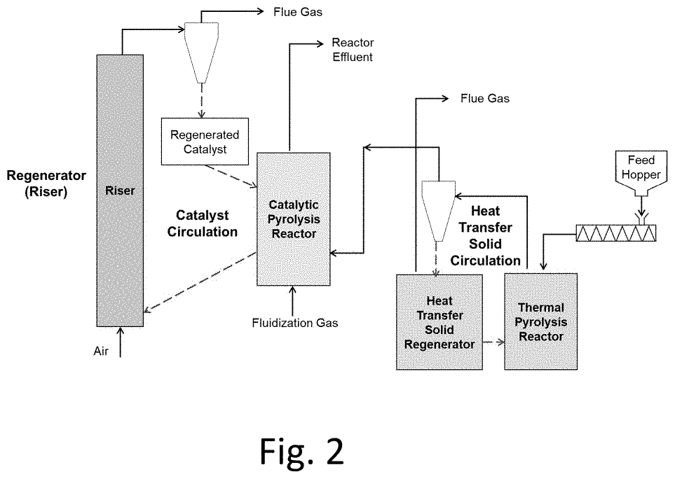

In some embodiments the feed material is thermally cracked in a thermal pyrolysis process and at least a portion of the products are fed to the catalytic pyrolysis reactor as vapor, as shown in FIG. 2 Error! Reference source not found. Products of the thermal pyrolysis could be also used as fluidization gas for the catalytic pyrolysis reactor and/or the thermal pyrolysis. The thermal cracking may be conducted in any type of fluid bed reactor, extruder, rotating kiln reactor, or other suitable reactor, that produces a vapor stream and a solid stream. A portion of the solids stream from the thermal cracking reaction may be combusted to provide energy for the process or discarded. In some embodiments the fluid bed thermal pyrolysis reactor comprises heat transfer materials that may be separated in one or more cyclones, regenerated in a regenerator, and a portion of the solids returned to the thermal pyrolysis reactor, as shown in FIG. 2 Error! Reference source not found.. The heat transfer materials can comprise an inert fluidizable material. The fluidization fluid for a fluidized bed thermal cracking reactor may be an inert gas, or a hydrocarbon gas, or a recycle stream separated from the products, or a combination thereof. In embodiments wherein an extruder, rotating kiln, or other reactor is used, the materials that are not converted to vapors are separated in the thermal pyrolysis step, and can be combusted to generate heat or energy, or discarded. These reactor types are advantageous where the feed comprises significant quantities of non-hydrocarbonaceous materials, such as fillers, inorganics, salts, minerals, or bits of glass or metal that are not separated in any feed preparation step(s).

In some embodiments that include a thermal pyrolysis step as in FIG. 2 Error! Reference source not found., the thermal pyrolysis reactor is operated at temperatures from 400 to 700, or from 450 to 650, or from 500 to 600, or at least 400, or at least 450, or at least 500, or no more than 700, or no more than 650, or no more than 600° C. In some embodiments at least a portion of the heat required for the thermal pyrolysis is provided by shear heating of the polymer from mechanical work or a mechanical/moving/rotating device, such as an extruder or other device, that induces shear heating within the molten plastic mixture.

In some embodiments the residence time of the solid feed materials in the thermal pyrolysis reactor is from 1 to 30, or from 2 to 20, or from 5 to 15, or at least 1, or at least 2, or at least 5, or no more than 30, or no more than 20 or no more than 15 minutes. In some embodiments the pressure in the thermal pyrolysis reactor can be from 1 to 30, from 2 to 20, from 3 to 10, or at least 2, or at least 3 or at least 4, or no more than 30, or no more than 20 or no more than 10 barg.

In another embodiment of the process, olefins are separated from the catalytic pyrolysis products for upgrading to BTX or other valuable products, and at least a portion of the olefins are recycled to the catalytic pyrolysis (Plas-TCat) reactor. This configuration of the inventive process takes advantage of the capability of the Plas-TCat process to convert olefins to aromatics, boosting the yield of aromatics obtained from the Plas-TCat reactor, and improving the efficiency of the overall process. In this embodiment of the invention the products of the Plas-TCat and olefins upgrading processes may be handled separately or combined for separation and purification into the desired high value products. In this embodiment the number of unit operations is minimized, and the capital investment is reduced compared to some other embodiments of the process, and this embodiment may be more applicable to stand-alone plants where opportunities for integration with nearby processes are not available.

### Glossary

Aromatics—As used herein, the terms “aromatics” or “aromatic compound” are used to refer to a hydrocarbon compound or compounds comprising one or more aromatic groups such as, for example, single aromatic ring systems (e.g., benzyl, phenyl, etc.) and fused polycyclic aromatic ring systems (e.g. naphthyl, 1,2,3,4-tetrahydronaphthyl, etc.). Examples of aromatic compounds include, but are not limited to, benzene, toluene, indane, indene, 2-ethyl toluene, 3-ethyl toluene, 4-ethyl toluene, trimethyl benzene (e.g., 1,3,5-trimethyl benzene, 1,2,4-trimethyl benzene, 1,2,3-trimethyl benzene, etc.), ethylbenzene, styrene, cumene, methylbenzene, propylbenzene, xylenes (e.g., p-xylene, m-xylene, o-xylene, etc.), naphthalene, methyl-naphthalene (e.g., 1-methyl naphthalene, anthracene, 9.10-dimethylanthracene, pyrene, phenanthrene, dimethyl-naphthalene (e.g., 1,5-dimethylnaphthalene, 1,6-dimethylnaphthalene, 2,5-dimethylnaphthalene, etc.), ethyl-naphthalene, hydrindene, methyl-hydrindene, and dymethyl-hydrindene. Single-ring and/or higher ring aromatics may also be produced in some embodiments.

Fluid—The term “fluid” refers to a gas, a liquid, a mixture of a gas and a liquid, or a gas or a liquid containing dispersed solids, liquid droplets and/or gaseous bubbles. The terms “gas” and “vapor” have the same meaning and are sometimes used interchangeably. In some embodiments, it may be advantageous to control the residence time of the fluidization fluid in the catalytic pyrolysis reactor. The fluidization residence time of the fluidization fluid is defined as the volume of the reactor divided by the volumetric flow rate of the fluidization fluid under process conditions of temperature and pressure. In embodiments comprising a thermal pyrolysis reactor before the catalytic pyrolysis, the residence time of the feed in the thermal pyrolysis reactor can be calculated by the feed rate of solid hydrocarbonaceous material divided by the heated reactor volume.

Fluidized Bed Reactor—The term “fluidized bed reactor” is given its conventional meaning in the art and is used to refer to reactors comprising a vessel that can contain a granular solid material (e.g., silica particles, catalyst particles, etc.), in which a fluid (e.g., a gas or a liquid) is passed through the granular solid material at velocities sufficiently high as to suspend the solid material and cause it to behave as though it were a fluid. The term “circulating fluidized bed reactor” is also given its conventional meaning in the art and is used to refer to fluidized bed reactors in which the granular solid material is passed out of the reactor, circulated through a line in fluid communication with the reactor, and recycled back into the reactor. Examples of fluidized bed reactors are described in “Fluidization Engineering”, D. Kunii and O. Levenspiel, 2nd Ed. Butterworth-Heinemann, 1991, pp 1-236.

Bubbling fluidized bed reactors and turbulent fluidized bed reactors are also known to those skilled in the art. In bubbling fluidized bed reactors, the fluid stream used to fluidize the granular solid material is operated at a sufficiently low flow rate such that bubbles and voids are observed within the volume of the fluidized bed during operation. In turbulent fluidized bed reactors, the flow rate of the fluidizing stream is higher than that employed in a bubbling fluidized bed reactor, and hence, bubbles and voids are not observed within the volume of the fluidized bed during operation. Examples of bubbling and turbulent fluidized bed reactors are described in “Fluidization Engineering”, D. Kunii and O. Levenspiel, 2^{nd} Ed. Butterworth-Heinemann, 1991, pp 1-236, incorporated herein by reference.

Olefins—The terms “olefin” or “olefin compound” (a.k.a. “alkenes”) are given their ordinary meaning in the art and are used to refer to any unsaturated hydrocarbon containing one or more pairs of carbon atoms linked by a double bond. Olefins include both cyclic and acyclic (aliphatic) olefins, in which the double bond is located between carbon atoms forming part of a cyclic (closed-ring) or of an open-chain grouping, respectively. In addition, olefins may include any suitable number of double bonds (e.g., monoolefins, diolefins, triolefins, etc.). Examples of olefin compounds include, but are not limited to, ethene, propene, allene (propadiene), 1-butene, 2-butene, isobutene (2 methyl propene), butadiene, and isoprene, among others. Examples of cyclic olefins include cyclopentene, cyclohexane, cycloheptene, among others. Aromatic compounds such as toluene are not considered olefins; however, olefins that include aromatic moieties are considered olefins, for example, benzyl acrylate or styrene.

Catalysts—Catalyst components useful in the context of this invention can be selected from any catalyst known in the art, or as would be understood by those skilled in the art. Catalysts promote and/or effect reactions. Thus, as used herein, catalysts lower the activation energy (increase the rate) of a chemical process, and/or improve the distribution of products or intermediates in a chemical reaction (for example, a shape selective catalyst). Examples of reactions that can be catalyzed include: dehydration, dehydrogenation, isomerization, hydrogen transfer, hydrogenation, polymerization, cyclization, desulfurization, denitrogenation, deoxygenation, aromatization, decarbonylation, decarboxylation, aldol condensation, and combinations thereof. Catalyst components can be considered acidic, neutral, or basic, as would be understood by those skilled in the art.

For catalytic pyrolysis, particularly advantageous catalysts include those containing internal porosity selected according to pore size (e.g., mesoporous and pore sizes typically associated with zeolites), e.g., average pore sizes of less than 10, less than 5, less than 2, less than 1, less than 0.65 nanometers, or smaller. In some embodiments, catalysts with average pore sizes of from 0.5 to 10 nanometers may be used. In some embodiments, catalysts with average pore sizes of between about 0.55 and 0.65, or between 0.59 and 0.63 nanometers may be used. In some cases, catalysts with average pore sizes of between 0.7 and 0.8, or between 0.72 and 0.78 nanometers may be used.

In some preferred embodiments of catalytic pyrolysis, the catalyst may be selected from naturally occurring zeolites, synthetic zeolites and combinations thereof. In certain embodiments, the catalyst may be a ZSM-5 zeolite catalyst, as would be understood by those skilled in the art, which can include the proton form of ZSM-5 sometimes written as HZSM-5, or any form wherein protons have been substituted at least in part with metal cations. Optionally, such a catalyst can comprise acidic sites. Other types of zeolite catalysts include: ferrierite, zeolite Y, zeolite beta, mordenite, MCM-22, ZSM-23, ZSM-57, SUZ-4, EU-1, ZSM-11, (S)A1PO-31, SSZ-23, among others. Zeolites and other small pore materials are often characterized by their Constraint Index (CI). The method by which Constraint Index is determined is described more fully in U.S. Pat. No. 4,029,716, incorporated by reference for details of the method. The CI preferably has a value for any given molecular sieve useful herein within the approximate range of 1 to 12.

In other embodiments, non-zeolite catalysts may be used; for example, WOx/ZrO₂, aluminum phosphates, etc. In some embodiments, the catalyst may comprise a metal and/or a metal oxide chosen from among nickel, palladium, platinum, titanium, vanadium, chromium, manganese, iron, cobalt, zinc, copper, gallium, the rare earth elements, i.e., elements 57-71, cerium, zirconium, and/or any of their oxides, or some combination thereof. In addition, in some cases, properties of the catalysts (e.g., pore structure, type and/or number of acid sites, etc.) may be chosen to selectively produce a desired product.

Plastics or Polymers—The terms “plastics” and “polymers” are used interchangeably herein. A polymer is a carbon-based (at least 35 mass % C) material chiefly made up of repeating units and having a number average molecular weight of at least 100, typically greater than 1000 or greater than 10,000.

Pyrolysis—The terms “pyrolysis” and “pyrolyzing” are given their conventional meaning in the art and are used to refer to the transformation of a compound, e.g., a solid hydrocarbonaceous material, into one or more other substances, e.g., volatile organic compounds, gases, and coke, by heat, preferably without the addition of, or in the absence of, 02. Preferably, the volume fraction of 02 present in a pyrolysis reaction chamber is 0.5% or less. Pyrolysis may take place with or without the use of a catalyst. “Catalytic pyrolysis” refers to pyrolysis performed in the presence of a catalyst and may involve steps as described in more detail below. Example of catalytic pyrolysis processes are outlined, for example, in Huber, G. W. et al, “Synthesis of Transportation Fuels from Biomass: Chemistry, Catalysts, and Engineering,” Chem. Rev. 106, (2006), pp. 4044-4098.

Selectivity—The term “selectivity” refers to the amount of production of a particular product in comparison to a selection of products. Selectivity to a product may be calculated by dividing the amount of the particular product by the amount of a number of products produced. For example, if 75 grams of aromatics are produced in a reaction and 20 grams of benzene are found in these aromatics, the selectivity to benzene amongst aromatic products is 20/75=26.7%. Selectivity can be calculated on a mass basis, as in the example, or it can be calculated on a carbon basis, where the selectivity is calculated by dividing the amount of carbon that is found in a particular product by the amount of carbon that is found in a selection of products. Unless specified otherwise, for reactions involving polymers as reactants, selectivity is on a mass basis. For reactions involving conversion of a specific molecular reactant (ethene, for example), selectivity is the percentage (on a mass basis unless specified otherwise) of a selected product divided by all the products produced.

Yield—The term yield is used herein to refer to the amount of a product flowing out of a reactor divided by the amount of reactant flowing into the reactor, usually expressed as a percentage or fraction. Yields are often calculated on a mass basis, carbon basis, or on the basis of a particular feed component. Mass yield is the mass of a particular product divided by the weight of feed used to prepare that product. For example, if 500 grams of polymer is fed to a reactor and 45 grams of benzene is produced, the mass yield of benzene would be 45/500=9% benzene. Carbon yield is the mass of carbon found in a particular product divided by the mass of carbon in the feed to the reactor. For example, if 500 grams of polymer that contains 90% carbon is reacted to produce 400 grams of benzene that contains 92.3% carbon, the carbon yield in benzene is [(400*0.923)/(500*0.90)]=82.0%.

As is standard patent terminology, the term “comprising” means “including” and does not exclude additional components. Any of the inventive aspects described in conjunction with the term “comprising” also include narrower embodiments in which the term “comprising” is replaced by the narrower terms “consisting essentially of” or “consisting of”. As used in this specification, the terms “include”, “includes” or “including” should not be read as limiting the invention but, rather, listing exemplary components.

Upflow and downflow. The terms ‘upflow’ and ‘downflow’ as used herein are defined with respect to the net direction of flow of the catalyst within the unit, i.e. where the inlet of the catalyst is positioned with respect to the outlet of the catalyst. Catalyst within a fluidized bed comprises catalyst particles that are moving in all directions due to the fluidization of the material, nevertheless if the catalyst enters the fluid bed at a level above that level at which it leaves the bed the direction of flow is defined as downflow, and if the catalyst enters the bed at a level below the level at which it leaves the bed the direction of flow is defined as upflow.

### DESCRIPTION OF SOME PREFERRED EMBODIMENTS

The various features, characteristics, embodiments, etc. that are described herein are not limited to a single aspect or embodiment and should be understood as applicable to any of the inventive aspects described herein.

In an embodiment of the invention, polymers or plastics or polymers and plastics are fed to a catalytic pyrolysis reactor to form a gaseous product containing aromatic compounds and olefins, wherein the olefins are separated from the product, the olefins are purified and separated into the various component olefins, and each olefin stream is sent for further processing for conversion to useful products, or recycled to the catalytic pyrolysis reactor.

The mixed polymer feed to the process comprises one or any combination selected from the following materials: biomass, polyethylene (PE), polypropylene (PP), polyacetylene, polybutylene, polyolefins, polyethylene terephthalate (PET), polybutylene terephthalate, copolyesters, polyester, polycarbonate, polyurethanes, polyamides, polystyrene (PS), polyacetal, epoxies, polycyanurates, polyacrylics, polyurea, vinyl esters, polyacrylonitrile, polyvinyl alcohol, polyvinylchloride (PVC), poly(methyl methacrylate) (PMMA), polyvinyl acetate, nylon, copolymers such as: ethylene-propylene, EPDM, acrylonitrile-butadiene-styrene (ABS), nitrile rubber, natural and synthetic rubber, tires, styrene-butadiene, styrene-acrylonitrile, styrene-isoprene, styrene-maleic anhydride, ethylene-vinyl acetate, nylon 12/6/66, filled polymers, polymer composites, polymer composites comprising natural fibers, plastic alloys, other polymeric materials, and polymers or plastics dissolved in a solvent. The feed materials can comprise materials obtained from polymer or plastic manufacturing processes as waste or discarded materials, post-consumer recycled polymer materials, materials separated from waste streams such as municipal solid waste (MSW), black liquor, wood waste, or other biologically produced materials. In some embodiments, the feed material can comprise from 30 to 100, or from 40 to 80, or from 45 to 70, or at least 30, or at least 40, or at least 45, or less than 99, or less than 95, or less than 80 percent by mass of a combination of polyethylene (PE) (sum of low- and high-density polyethylene), polypropylene (PP), and polystyrene (PS). In some embodiments, the feed material can comprise from 1 to 30, or 2 to 20, or 3 to 10, or up to 30, or up to 20 or up to 10, or at least 0.1, or at least 1, or at least 2 percent by mass biomass. In some embodiments the feed can comprise from 0.1 to 20, or from 1 to 15, or from 3 to 10, or at least 0.1, or at least 1, or at least 3, or less than 20, or less than 15, or less than 10 percent by mass PET. In some embodiments the feed can comprise from 0.1 to 20, or from 1 to 15, or from 3 to 10, or at least 0.1, or at least 1, or at least 3, or less than 20, or less than 15, or less than 10 percent by mass nylon.

In some embodiments the feed materials used in the process are pretreated at least in part to reduce contaminant concentrations in a contaminant removal process before addition to the catalytic pyrolysis process. A “contaminant” is a material such as silica or metal or metal oxide or salt or carbon black or clay or any other material that is commonly used as an additive in commercially available plastics or an element that may poison the catalyst or contribute to a reduction in catalyst activity, that does not pyrolyze under typical pyrolysis conditions. Removal of contaminants from plastics can be accomplished by filtering off solids from a solution or melt, or in some preferred embodiments, by a first pyrolysis step, without added catalyst (or without zeolite catalyst). The contaminant removal process can include any of those such as washing with water or a solvent, and by those described in U.S. Pat. Nos. 10,336,628, 6,792,881, 7,303,649, 7,503,981, 8,101,024, U.S. Pat. Nos. 9,109,049, 9,468,950, and US Patent Application Publication US 2015/0166683, or any method known to those skilled in the art.

In some embodiments, it may be advantageous to feed the polymers at least in part as molten material. This can be done with polymers or plastics alone or as mixtures of polymers and plastics that melt at temperatures below 200° C. In some embodiments the molten polymers may be atomized before entrance into the pyrolysis reactor. This can be done with a carrier gas input or gas mixture recycled from the pyrolysis product separation section. Gas mixtures can comprise argon, helium, nitrogen, carbon dioxide, carbon monoxide, hydrogen, methane, ethane, propane, butane, ethylene, propylene, or mixtures of these.

In some embodiments the molten mixture of polymers, or plastics, or polymers and plastics may be filtered to remove solids that do not readily melt at the chosen process conditions using any of the variety of filtering procedures known to those skilled in the art. In some embodiments in which the molten mixture of polymers, or plastics, or polymers and plastics, comprises materials that contain carbonaceous solids, these solids may be separated by hot filtration and optionally combusted to provide energy for the process.

The catalytic pyrolysis reactor comprises a fluidized bed reactor, such as a bubbling bed or turbulent bed reactor. Fluidized bed reactors may, in some cases, provide improved mixing of the catalyst and/or polymeric material during pyrolysis and/or subsequent reactions, which may lead to enhanced control over the reaction products formed. The use of fluidized bed reactors may also lead to improved heat transfer within the reactor. In addition, improved mixing in a fluidized bed reactor may lead to a reduction of the amount of coke adhered to the catalyst, resulting in reduced deactivation of the catalyst in some cases and higher yields of olefins and other desirable products. Throughout this specification, various compositions are referred to as process streams; however, it should be understood that the processes could also be conducted in batch mode.

The catalytic pyrolysis reactor is a fluidized bed reactor; but the catalyst traverses the reactor in a downwards direction such that the regenerated and any fresh catalyst fed to the reactor enters nearer the top of the reactor, and catalyst exits the reactor nearer the bottom of the reactor. The fresh catalyst may be fed with the regenerated catalyst or from a separate conduit or some combination of these. The reactor comprises a sparger or distributor, located at or near the bottom of the reactor, that serves to distribute the fluidization fluid. The fluid bed is fluidized with a fluidization fluid such as an inert gas, or a hydrocarbon gas, or a recycle stream separated from the products, or some combination of these, at a velocity and density sufficient to induce operation in the bubbling or turbulent flow regime in the catalyst bed, as described in “Fluidization Engineering”, D. Kunii and O. Levenspiel, 2nd Ed. Butterworth-Heinemann, 1991, pp 1-236. The catalytic pyrolysis reactor operates in either the bubbling or turbulent fluidization regimes, or a combination of both due to axial increase in gas velocity resulting from gas generation from the thermal pyrolysis of feed materials. The inlet gas velocity of the fluidization fluid entering the reactor can range from 0.05 to 1.0, or from 0.1 to 0.7, or from 0.2 to 0.5, or at least 0.1, or at least 0.2, or at least 0.3, or less than 1.0, or less than 0.7, or less than 0.5 meters per second.

The residence time of the feed material within the reactor is an important feature that impacts the performance of the system, the conversion of the feed, and the selectivity to different products. For the catalytic pyrolysis process of this invention the residence time of an average carbon atom (meaning averaged over all carbon entering the reactor) of the solid hydrocarbonaceous materials within the catalytic pyrolysis reactor can range from 0.5 to 180, or from 2 to 100, or from 4 to 80, or from 10 to 60, or from 30 to 60, or at least 5, or at least 10, or at least 30, or less than 180, or less than 100, or less than 80, or less than 60 seconds. The residence time is calculated as the average time a carbon atom spends in the reactor at a temperature of at least 400° C. under actual conditions of temperature and pressure.

The weight hourly space velocity is another factor that greatly impacts the performance of the process by affecting the yield and selectivity of various valuable products and the operability of the system. The weight hourly space velocity is defined as the mass rate of feed of solid hydrocarbonaceous materials divided by the mass of catalyst in the reactor bed:

$$WHSV ( 1 / hr ) = \frac{{\dot{m}}_{feed} ( kg / hr )}{Weight of catalyst in bed ( kg )}$$

In some embodiments of the inventive process the WHSV can range from 0.1 to 2.0, or from 0.2 to 1.0, or from 0.25 to 0.75, or at least 0.1, or at least 0.2, or at least 0.25, or less than 2.0, or less than 1.0, or less than 0.75 hr⁻¹.

The catalytic pyrolysis reactor can be any size suitable for conversion of solid materials to higher value products. In some embodiments the capacity of the reactor will be suitable for processing 5 to 5000 metric tons per day (mtpd) of feed, or from 5 to 2500 mtpd, or 100 to 1500 mtpd, or at least 25 mtpd, or at least 100 mtpd of feed materials. The diameter of the catalytic pyrolysis reactor can range from 0.1 to 15, or from 0.3 to 10, or from 3 to 7, or at least 3, or at least 5 meters as required to meet the demands of the rate of introduction of feed materials; the required diameter increases as approximately the square root of the increase in capacity of the reactor. The catalytic pyrolysis reactor is characterized by a height/diameter ratio (H:D) that can be from 0.5:1 to 10:1, or from 1:1 to 8:1, or from 1.5:1 to 5:1, or at least 1:1, or at least 1.5:1, or at least 2:1, or less than 15:1, or less than 10:1, or less than 5:1. In some cases, the reactor has a uniform internal diameter. In cases where the diameter is not uniform, “diameter” refers to the diameter averaged over the height of the reactor.

The temperature of the catalytic pyrolysis reactor can be maintained at a temperature that is high enough to provide high conversion of the feed materials and yet low enough to not produce a very high yield of coke and char. The temperature for the catalytic pyrolysis reactor can be from 450 to 750, or from 500 to 650, or from 550 to 600, or at least 450, or at least 500 or at least 550, or no more than 750, or no more than 650° C. Heat is supplied to the catalytic pyrolysis reactor by the feed of hot, regenerated catalyst to the reactor. The mass flow rate of the hot, regenerated catalyst recycle stream is adjusted to provide enough heat to maintain the reactor temperature and drive the thermal pyrolysis reaction. The mass flow rate of the hot, regenerated catalyst recycle stream can be from 3 to 1700, or from 170 to 845, or at least 60, or at least 150 or at least 310, or less than 170, or less than 360 or less than 845 kg/s, as required to meet the heat demands of the reactor. Alternatively, the mass flow rate of the hot regenerated catalyst into the catalytic pyrolysis reactor, when defined as the percent of catalyst added per unit time divided by the amount of catalyst in the bed, can be from 0.025% per second to 5% per second, or from 0.1% per second to 3.5% per second, or at least 0.5% per second, or at least 1.5% per second, or at least 3% per second, or less than 7% per second, as required to meet the heat demands of the reactor. Additional heat may be provided from other sources to maintain the desired temperature of the process if needed.

In some embodiments a portion of the regenerated catalyst is removed from the separated stream of regenerated catalyst and discarded. In some embodiments the amount of catalyst that is removed and discarded can be from 0.1 to 5.0, or from 0.5 to 4.0, or from 1.0 to 3.0, or at least 0.1, or at least 0.5, or at least 1, or no more than 5.0, or no more than 4.0 or no more than 3.0% by mass of the regenerated catalyst each day. In some embodiments fresh catalyst may be fed to the catalytic pyrolysis reactor with the regenerated catalyst or from a separate conduit or some combination of these. In some embodiments the amount of fresh catalyst added to the catalytic pyrolysis reactor can be from 0.1 to 5.0, or from 0.5 to 4.0, or from 1.0 to 3.0, or at least 0.1, or at least 0.5, or at least 1, or no more than 5.0, or no more than 4.0 or no more than 3.0% by mass of the regenerated catalyst each day.

The pressure of the catalytic pyrolysis process can be maintained at a pressure that is high enough to provide sufficient driving force to move the materials through the apparatus, and yet low enough to maintain good fluidization, minimize compression costs, and allow for facile feed of feed materials and catalyst. Suitable pressures are from 0.5 to 10, or 1.0 to 6, or 1.5 to 4, or at least 1, or at least 1.5 or at least 2, or less than 20, or less than 10, or less than 6 barg.

As material is pyrolyzed and catalytically converted in the catalytic pyrolysis reactor, gases are evolved that increase the volume of the gas since more than one mole of gaseous products is produced from a mole of reacting feed material. In this situation with a cylindrical reactor, the superficial gas velocity increases as the gaseous products from both thermal and catalytic pyrolysis flows upward through the reactor. The superficial gas velocity will increase axially along the height of the reactor, depending on the nature of the feed, the performance of the catalyst, and the reactor diameter. The increase in superficial gas velocity between the bottom and top of the cylindrical reactor will be in the range of 1.5 and 8 times, depending on conditions, feed, etc. This gas volume expansion reduces the residence time of the feed in the reactor, reduces gas-solid contact, reduces conversion, and in the extreme can transport catalyst out of the top of the reactor.

A pyrolysis reactor with a larger diameter at higher levels has the advantage of having a more uniform gas velocity along the height of the reactor which increases BTX and olefins yield and reactor productivity compared to a cylindrical shaped reactor in which gas velocity increases along the height of the reactor. Reactor performance is improved by decreasing the velocity gradient along the reactor. To overcome this deficiency and provide for a more uniform linear velocity of the feed materials and catalyst as the gas passes upwards through the fluid bed, the diameter of the reactor can increase along the height of the reactor. A conical geometry with an expanding diameter along the height of the reactor to accommodate the higher gas generation provides better performance.

In some embodiments the diameter of the catalytic pyrolysis reactor increases along at least a portion of the height of the reactor. In these embodiments the diameter of the portion of the reactor that increases is smaller nearer the bottom of the reactor and larger nearer the top of the reactor. The variable diameter of at least a portion of the reactor can be shaped like a cone where the angle of the wall of the cone with respect to vertical can range from 3 degrees to 50 degrees, or from 5 to 40, or from 7 to 25, or from 8 to 15, or at least 3, or at least 7, or at least 8 degrees, or at least 10 degrees from vertical (vertical is defined as zero degrees). In some embodiments the ratio of the superficial velocity of the vapors at the top of the conical portion of the reactor is no more than 1.01:1, or no more than 1.5:1, or no more than 2.0:1, or no more than 2.5:1, or no more than 3.0:1, or from 1.01:1 to 3.0:1, or from 1.1:1 to 2.0:1 compared to the superficial velocity of the vapors at the bottom of the conical portion of the reactor. The conical portion of the reactor should include at least the height of the dense bubbling phase of the fluid bed. Any additional height of the reactor above the top of the fluid bed, the so-called ‘freeboard,’ can be cylindrical as very little reaction occurs in this zone so the gas expansion is negligible.

Catalyst may be removed from the catalytic pyrolysis reactor via a solids exit conduit such as an overflow standpipe or drainpipe or other means. In some cases, the catalyst removed from the catalytic pyrolysis reactor may be at least partially deactivated. The catalyst removed from the catalytic pyrolysis reactor may be fed to a regenerator in which catalyst that was at least partially deactivated may be reactivated. The regenerator may comprise an optional purge stream, which may be used to purge solids such as coke, ash, catalyst, or some combination of these, from the regenerator.

The catalyst regenerator may be of any suitable size mentioned above in connection with the reactor or the solids separator. In addition, the regenerator may be operated at elevated temperatures e.g., at least 300, 400, 500, 600, 750, 800, or higher, or from 500 to 1000, or from 600 to 800, or from 650 to 750° C. The residence time of the catalyst in the regenerator may also be controlled using methods known by those skilled in the art, including those outlined above. The residence time of the catalyst in the regenerator can be from 25 to 500, or from 50 to 300, or from 75 to 150, or at least 25, or at least 50, or at least 75, or at least 100, or no more than 500, or no more than 300 or no more than 150 seconds. The mass flow rate of the catalyst through the regenerator may be coupled to the flow rate(s) in the reactor and/or solids separator to preserve the mass balance in the system.

An oxidizing agent can be fed to the regenerator via a gas feed stream. The oxidizing agent may originate from any source including, for example, a source of oxygen, air, recycled flue gas, or steam, or some combination of these, among others. The regenerator comprises a sparger or distributor, located at or near the bottom of the regenerator, that serves to distribute the fluidization fluid. The fluid bed is fluidized with a fluidization fluid such as air, or oxygen in nitrogen, or flue gas, or steam, or some combination of these, at a velocity and density sufficient to induce operation in the fast fluidization regime in the regenerator bed, and pneumatic transport of the catalyst to the top of the bed, as described in “Fluidization Engineering”, D. Kunii and O. Levenspiel, 2nd Ed. Butterworth-Heinemann, 1991, pp 73-94 and pp 359-396. In some embodiments the superficial fluidization gas velocity at the entrance of the regenerator can be at least 1.5, or at least 2.0, or at least 2.5, or at least 3.0, or from 0.6 to 7.5, or from 1.0 to 6.0, or from 1.5 to 5.0, or from 2.5 to 4.5, or from 3.0 to 4.0, or no more than 7.5, or no more than 6.5, or no more than 5.0, or no more than 4.0 m/s. In some embodiments the height to diameter ratio (H/D) of the regenerator can be from 2 to 15, or from 3 to 22, or from 5 to 40, or from 10 to 44, or at least 2, or at least 5, or at least 10, or no more than 10, or no more than 15, or no more than 30, or no more than 44.

In the regenerator, the catalyst is re-activated by reacting the catalyst with the oxidizing agent. In some cases, the deactivated catalyst may comprise residual carbon and/or coke, which may be removed via reaction with the oxidizing agent in the regenerator. The regenerator comprises a vent stream which may include regeneration reaction products, residual oxidizing agent, etc. The exhaust gas vent stream from the regenerator may be passed through a catalytic exhaust gas cleanup system to further reduce the concentrations of CO and hydrocarbons to reduce emissions vented to the atmosphere. Portions of the exhaust gas vent stream may be recycled to the gas feed of the regenerator to control the heat release of the regeneration process. A separated stream of gases comprising gases chosen from among any of C1-C4 hydrocarbons, H₂, and CO can be separated from the vapor products and at least a portion of the separated stream of gases is passed to the regenerator as part of the fluidization fluid.

The regeneration may be operated with fluidization flow rates suitable to maintain continuous transport of the catalyst in an upflow direction within the regenerator. The flow velocity of the gas mixture within the regenerator is affected by many parameters including: the mass flow of catalyst, the size and density of catalyst particles, the amount of coke and char and other materials that will be combusted in the regenerator, the feed mixture to the catalyst pyrolysis unit, the system pressure, and the temperature profile of the unit. The solids flux, which is the rate of mass flow of solid material through a cross sectional area of the regenerator, is in the range from 19 to 300, or 50 to 250, or 100 to 200, or at least 19 or at least 50, or at least 100, or at least 150, or no more than 300, or no more than 250, or no more than 200 kg/m²s.

The reaction products from the catalytic pyrolysis reactor (e.g., fluid hydrocarbon products) may be fed to a solids separator where solid catalyst may be separated from the fluid products. In some instances, the initial products of the process may be fed to a quench tower to which is fed a cooling fluid, preferably a liquid, along with the product stream to cool and condense the products. In some embodiments, the desired reaction product(s) (e.g., liquid aromatic hydrocarbons, olefin hydrocarbons, gaseous products, etc.) may be recovered at any point in the production process (e.g., after passage through the reactor, after separation, after condensation, etc.).

The reaction products may be quenched to remove heavy hydrocarbons into a quench fluid. The quench fluid may comprise liquid products recovered in subsequent separation steps. The gaseous stream from the quench can be sent to a fractionation tower where the various aromatic liquid components can be recovered. The gaseous stream from the top of the fractionation tower can be sent to an absorption tower where the final fraction of remaining liquid organic is recovered. This can be done using a lean oil fraction as the absorption fluid which can comprise liquid product recovered from the fractionation tower or other available liquid known to those skilled in the art. A portion of the gaseous stream, now stripped of most higher boiling products, may be sent back to the reactor as fluidization fluid or for further conversion. The balance of the gaseous stream can be sent to a product recovery system for purification of the olefins. The recovered olefins can be sent to any of a variety of upgrading processes to convert them to other products.

The molecular sieve for use herein or the catalyst composition comprising same may be thermally treated at high temperatures. This thermal treatment is generally performed by heating at a temperature of at least 370° C. for a least 1 minute and generally not longer than 20 hours (typically in an oxygen containing atmosphere, preferably air). While subatmospheric pressure can be employed for the thermal treatment, atmospheric pressure is desired for reasons of convenience. The thermal treatment can be performed at a temperature up to 925° C. The thermally treated product is particularly useful in the present process.

For the catalyst composition useful in the catalytic pyrolysis of this invention, the suitable molecular sieve may be employed in combination with a support or binder material such as, for example, a porous inorganic oxide support or a clay binder. Non-limiting examples of such binder materials include alumina, zirconia, silica, magnesia, thoria, titania, boria and combinations thereof, generally in the form of dried inorganic oxide gels and gelatinous precipitates. Suitable clay materials include, by way of example, bentonite, kieselguhr and combinations thereof. The relative proportion of suitable crystalline molecular sieve of the total catalyst composition may vary widely with the molecular sieve content ranging from 30 to 90 percent by weight and more usually in the range of 40 to 70 percent by weight of the composition. The catalyst composition may be in the form of an extrudate, beads or fluidizable microspheres.

The molecular sieve for use herein or the catalyst composition comprising it may have original cations replaced, in accordance with techniques well known in the art, at least in part, by ion exchange with hydrogen or hydrogen precursor cations and/or non-noble metal ions of Group VIII of the Periodic Table, i.e. nickel, iron and/or cobalt.

The volatile product stream from the catalytic pyrolysis reactor (the raw feed from the catalytic pyrolysis reactor, prior to purification) can comprise C2-C4 alkenes, including ethylene, propylene, butylenes, or butadienes, or combinations thereof. The vapor products from the catalytic pyrolysis can comprise from 1-70 wt %, or 5-65 wt %, or 10-60 wt %, or 20-50 wt %, or 30-45 wt %, or 40-65 wt %, or 50-70 wt %, or at least 20 wt %, or at least 30 wt %, or at least 40 wt %, or at least 50 wt %, or at least 60 wt % C2-C4 alkenes. In some embodiments a stream enriched in ethylene or propylene, or both is separated from the condensable higher materials in the vapor products. The mass ratio of ethylene to propylene can vary from 0.2 to 3 depending on reaction conditions and feedstock. The mass ratio of butenes to propylene can vary between 0.05 and 0.62. Other minor components such as C5-C7 olefins are present in much smaller mass ratios to propylene and can vary in the range of 0.05 to 0.25.

In broader aspects of the invention, the olefin-containing product stream separated from the condensable higher materials, can have a wide variety of compositions. The fraction could simply be the gaseous (noncondensed) fraction that includes CO, CO₂, ethylene, propylene, and numerous other components and may include higher olefins. The olefin-containing product could also contain alkynes such as ethyne, propyne, butyne or the like. Alternatively, the fraction could be a relatively olefin-rich stream that is separated from a relatively olefin-poor stream. Examples of separation techniques that can be used in a polymer conversion system include: cryogenic separation, distillation, membrane separation, adsorptive separation, or reactive separation. An olefin-containing product that is separated from the condensable higher materials in the vapor products comprises at least 20, or at least 50, or at least 70, or in the range of 20 to 95, or 50 to 90, or 70 to 90 mass % olefins, or more. Other gases in the olefin-containing product could include methane, ethane, propane, butanes, CO, CO2, water, propadiene, methyl acetylene, H2, or N2, or some combination thereof.

In some embodiments the mass yield of olefins is at least 1%, or at least 2.5%, or at least 5%, or at least 8%, or at least 9%, or no more than 40%, or no more than 25%, or no more than 15%, or from 1% to 40%, or from 3% to 28%, or from 5% to 15%, and the mass yield of all products is no more than 100% based on the mass of solid hydrocarbonaceous materials fed. A stream comprising C5+ products can be separated from the vapor products. In some embodiments, a stream comprising benzene, toluene, xylenes, or some combination of these (BTX) is separated from the vapor products. Mass yield of BTX may be at least 16%, or at least 22%, or at least 30%, or at least 40%, or at least 50%, or at least 55%, or at least 60%, or from 15% to 75%, or from 20% to 70%, or from 45% to 65%, and the mass yield of all products is no more than 100% based on the mass of solid hydrocarbonaceous materials fed to the process. The mass yield of coke and char may be less than 10%, or less than 5%, or less than 2%, or less than 1%, or less than 0.5%, or from 0.1% to 10%, or from 0.2% to 5%, or from 0.3 to 2%, and the mass yield of all products is no more than 100% based on the mass of solid hydrocarbonaceous materials fed. The mass yield of olefins plus aromatics can be greater than 40%, or greater than 60% or greater than 70%, or greater than 75%, or greater than 80%, or from 40% to 99%, or from 60% to 95%, or from 65% to 90%, and the mass yield of all products is no more than 100% based on the mass of solid hydrocarbonaceous materials fed to the process. In some embodiments, the selectivity of ethylene as a percentage of the total olefins produced is at least 20%, or at least 25%, or at least 30%, or from 10% to 60%, or from 20% to 45%, or from 25% to 35%, and the selectivity of propylene as a percentage of the total olefins produced is at least 20%, or at least 30%, or at least 40%, or at least 45%, or at least 50%, or from 20% to 70%, or from 25% to 65%, or from 28% to 55%, such that the total selectivity of ethylene plus propylene is less than 100%. In some embodiments the selectivity of benzene plus toluene plus xylenes as a percentage of aromatics produced is at least 40%, or at least 50%, or at least 80%, or at least 90%, or at least 95%, or at least 97%, or from 40% to 99.9%, or from 50% to 99.5%, or from 80 to 99%, or from 95% to 98%.

Olefin mixtures produced by the inventive process can be separated and purified by conventional cryogenic distillation, membrane separation, hybrid membrane distillation, selective adsorption, or facilitated transport systems as are known in the art. Impurities such as CO₂, HCl, HCN, or H₂S can be removed by amine scrubbing or caustic scrubbing or other conventional means known to those skilled in the art. Removal of impurities can be optionally performed before or after the separation of the olefins from the other vapor components.

Aromatics mixtures produced by the inventive process can be separated and purified by conventional distillation, membrane separation, hybrid membrane distillation, selective adsorption, or facilitated transport systems as are known in the art. Impurities such as phenols, thiols, thiophenes, nitriles, amines, or other oxygen, sulfur, or nitrogen containing impurities can be removed by hydrotreating or other conventional means known to those skilled in the art. Removal of impurities can be optionally performed before or after the separation of the aromatics from the other condensable components.

The catalytic pyrolysis process is normally conducted in an atmosphere with very low or zero oxygen (O₂) concentration, usually less than 0.5% by volume. Nevertheless, in some embodiments the pyrolysis process can be conducted with concentrations of O₂ of 0.6% by volume or greater in order to rapidly increase the temperature of the mixture to the desired reaction temperature, or to overcome the endothermic nature of the process, or both. In some embodiments the process feed is introduced at temperatures from 100° C. to 450° C. The temperature in the reactor can be rapidly increased by at least 25° C., or at least 100° C., or at least 200° C., or at least 300° C., or from 100° C. to 400° C. using small concentrations of O₂ in the process. The introduction of oxygen initiates combustion of hydrocarbons, CO, H₂, or other components, or some combination, in the process to supply the needed thermal energy to achieve conversion of the feed materials. In these cases the concentration of O₂ in the feed to the reactor resulting from this addition could be from 0.6% to 10%, 0.6% to 8%, 1% to 6%, or from 2% to 4% by weight, or at least 0.6%, at least 2%, at least 4%, or at least 6% by weight, where the percent weight of O₂ is in comparison to the weight of the polymer feed mixture, but in all cases the oxygen concentration introduced is kept below the concentration where significant unconverted oxygen (no greater than 0.2 mass %, preferably 0.1% or less, of the vapor products) may be found in the product mixture exiting the reactor. The oxygen is preferably introduced by the addition of air or O₂ as a component of the fluidization fluid, or with the gas injected with the plastics, or by separate, direct injection into the fluidized bed, or a combination thereof.

In any of the processes described herein, the olefin-containing gas can be partially separated into different fractions for functionalization, to remove non-reactive components, or to purge excess materials.

### Example 1

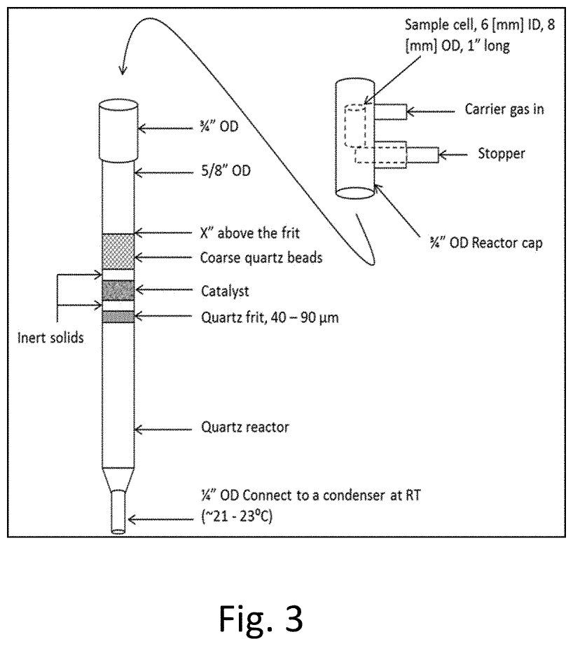

Drop-tube experiments were conducted to determine the relative product distributions produced for different feedstocks over a ZSM-5 catalyst in a fixed bed. The drop-tube reactor comprises a quartz reactor tube (ACE Glass) containing a quartz frit (40-90 μm) fused into the center of the tube. FIG. 3 FIG. 1 shows the configuration of the drop-tube reactor. A sample cell (10 mm OD, 8 mm ID, 25 mm length, quartz, made by TGP) is used to contain the feedstock using two pieces of quartz wool (TGP). As illustrated in FIG. 3 FIG. 1, the sample cell was placed in a reactor cap (borosilicate, ACE Glass) and was held by a stopper (¼ inch (6 mm) aluminum rod, McMaster). The reactor cap and the quartz reactor were then assembled and installed onto the fixed-bed reactor system. The bottom of the reactor was connected to a condenser (borosilicate) filled with perforated stainless steel packing (ACE Glass) immersed in an ice-water bath (0° C.). A heating mantle was applied between the reactor bottom and the condenser top to prevent any condensation before the condenser. During the reaction, the heating mantle was set at 210° C.

In the reactor, a small sample of ZSM-5 catalyst (1.5 g) was placed on top of the quartz frit. Feedstock (100 mg for each run) was sealed in a sample cell with the quartz wool. The catalyst/feedstock weight ratio was about 15. Prior to dropping the contents of the sample cell into the reactor, the catalyst was calcined at 550° C. under 100 mL/min air flow for 20 min (ramping rate=12° C./min). After calcination, the reactor was cooled to reaction temperature (500° C. for plastics and 525° C. for biomass). During the cool-down, the condenser was filled with 10 mL of solvent (ethyl acetate for plastics conversion, and acetone for biomass conversion) and held for 10 min for temperature lineout. The reactor system was then purged with helium flow at 75 mL/min for 20 min to remove air and to purge the gas collection lines. The sample cell was dropped into the reactor by pulling out the stopper rod to initiate the reaction.

A hold period of 10 min allowed the reaction to complete. Gas products, consisting mostly of permanent gases and C₁-C₃ olefins and paraffins were collected in a gas bag. Liquid products (mostly C₄₊) were collected in the condenser. After reaction the temperature was increased to 650° C. without gas flow. Solid products, including coke and char remaining in the reactor, were then burned at 650° C. for 10 min under 50 mL/min air flow. The gas products during burning were collected in a second gas bag. An additional 3 mL of solvent was added to the condenser to extract any products remaining on the top of the condenser. All of the liquid in the condenser was then transferred to a 20 mL sample vial. A weighed amount of internal standard (dioxane, typically 3000-5000 mg, Sigma-Aldrich) was added to the sample vial. The condenser was washed with acetone and was dried in a drying oven. It is noted that a small amount of liquid was retained in the condenser due to holdup on the packing. Therefore, the weight of the condenser with and without liquid products was measured to obtain the total amount of liquid products. Liquid samples were analyzed by a GC-FID (gas chromatograph with flame ionization detector from Shimadzu 2010Plus) for hydrocarbons and oxygenates. Gas bag samples were analyzed using an Agilent GC 7890B gas chromatograph.

The results of the experiments for various feeds are presented in TABLE 1, which summarizes products of catalytic pyrolysis of various materials with ZSM-5 catalyst in drop tube experiments. The balances of the products unaccounted for in TABLE 1 comprise water, inert solids, and minor components not readily recovered for combustion. All values are weight percent.

FIG. 1

TABLE 1

| | | Other | | | | | | | | |
|---|---|---|---|---|---|---|---|---|---|---|
| | | C5+ | Coke | | | | | | | |
| Feed | BTX | Liquid | and Char | Olefins | Paraffins | H2 | CO | CO2 | Ash | Total |
| Polyethylene (PE) | 52.8 | 3.5 | 0.9 | 18.1 | 16.4 | 2.6 | 0.3 | 0.3 | 0 | 94.8 |
| Polypropylene (PP) | 45.6 | 4.0 | 0.7 | 21.5 | 16.5 | 2.4 | 0 | 0.2 | 0 | 90.8 |
| High Density | 53.05 | 3.47 | 1.05 | 17.25 | 18.53 | 2.7 | 0 | 0.21 | 0 | 96.3 |
| Polyethylene (HDPE) | | | | | | | | | | |
| Isoprene | 32.88 | 10.03 | 1.29 | 10.43 | 5.89 | 1.29 | 0 | 0.28 | 0 | 62.1 |
| Tire Side wall | 14.94 | 3.13 | 21.62 | 8.25 | 2.08 | 0.9 | 0.56 | 0.95 | 7.0 | 59.4 |
| Tire Tread | 13.9 | 2.99 | 20.37 | 7.53 | 2.12 | 0.94 | 0.25 | 0.8 | 7.0 | 55.9 |
| Biomass | 5.96 | 1.34 | 23.69 | 2.95 | 2.66 | 0.58 | 17.42 | 9.4 | 0.01 | 64.0 |
| Cellulose | 4.8 | 1.13 | 18.74 | 2.76 | 2.06 | 0.42 | 13 | 13 | 0 | 55.9 |
| Cotton Clothing | 5.97 | 1.32 | 16.68 | 3.47 | 2.02 | 0.49 | 14.62 | 15.72 | 0 | 60.3 |
| PET | 23.73 | 3.92 | 17.76 | 4.98 | 1.16 | 0.38 | 7.65 | 34.13 | 0 | 93.7 |
| PET Clothing | 23.06 | 4.12 | 16.96 | 2.17 | 1.3 | 0.38 | 7.37 | 31.67 | 0 | 87.0 |
| Cellulose Acetate | 7.85 | 1.45 | 14.99 | 5.44 | 2.62 | 0.41 | 9.58 | 25.31 | 0 | 67.7 |
| Polystyrene (PS) | 38.51 | 36.29 | 4.13 | 5.58 | 1.49 | 0.48 | 0 | 0.33 | 0 | 86.8 |
| Nylon | 10.08 | 6.2 | 10.0 | 16.57 | 1.23 | 1.2 | 3.72 | 4.19 | 0 | 53.2 |

### Example 2

A computational model was developed to represent the process of catalytic pyrolysis in a fluidized bed reactor. The kinetics of plastic thermal pyrolysis for the various components in the feed were adapted from Zhao, Dongting, et al. “The chemistry and kinetics of polyethylene pyrolysis: a process to produce fuels and chemicals.” ChemSusChem 13.7 (2020): 1764-1774 and from Bockhorn, H., A. Hornung, and U. Hornung. “Stepwise pyrolysis for raw material recovery from plastic waste.” Journal of Analytical and Applied Pyrolysis 46.1 (1998): 1-13, and the kinetics of the catalytic conversion of the pyrolysis products in the reactor were derived from experimental data measured in a drop tube setup under experimental conditions relevant to the process conditions and using representative feedstocks.

The computational model used a nominal feed rate of 500 mtpd of plastics that include PE, PP, PS, PET, Nylon, ABS, and PMMA (poly methyl methacrylate). The mixtures of plastics evaluated are summarized in TABLE 2. The polymer mixture density was assumed to be 1 g/cc and the particle diameter was set to 3 mm. The reactor temperature was set to 500° C., the regenerator temperature was set at 650° C., and the reactor pressure was 4 bar gauge. The catalyst was assumed to have a density of 1.3 g/cc, a diameter of 100 microns, a specific heat at 650° C. or 1.167 kJ/kg-K and at 500° C. of 1.027 kJ/kg-K.

TABLE 2

| | Plastic, wt % | | |
|---|---|---|---|
| | High PO | Nominal case | Low PO |
| PE | 67 | 38 | 30 |
| PP | 10 | 16 | 18 |
| PS | 13 | 17 | 10 |
| PET | 3 | 9 | 14 |
| Nylon | 4 | 12 | 18 |
| ABS | 1.5 | 4 | 5 |
| PMMA | 1.5 | 4 | 5 |

The process was evaluated for four different conditions, WHSV=0.5 and 0.75, and inlet superficial velocity of fluidization gas of 0.15 and 0.3 m/s, all with a 3 meter diameter cylindrical reactor.

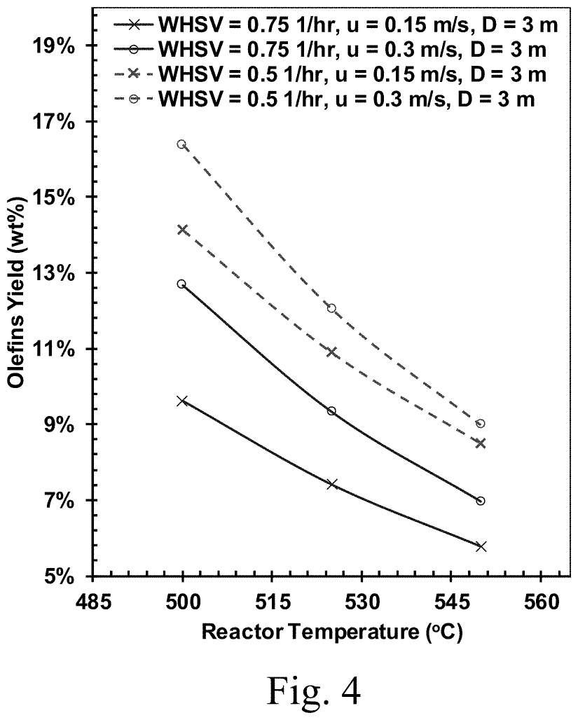

Error! Reference source not found. FIG. 4 shows the impact of temperature on the yield of olefins for the four different feed conditions. The data in FIG. 4 Error! Reference source not found. show that lower temperatures, lower space velocity, and higher initial superficial gas velocity favor olefins formation.

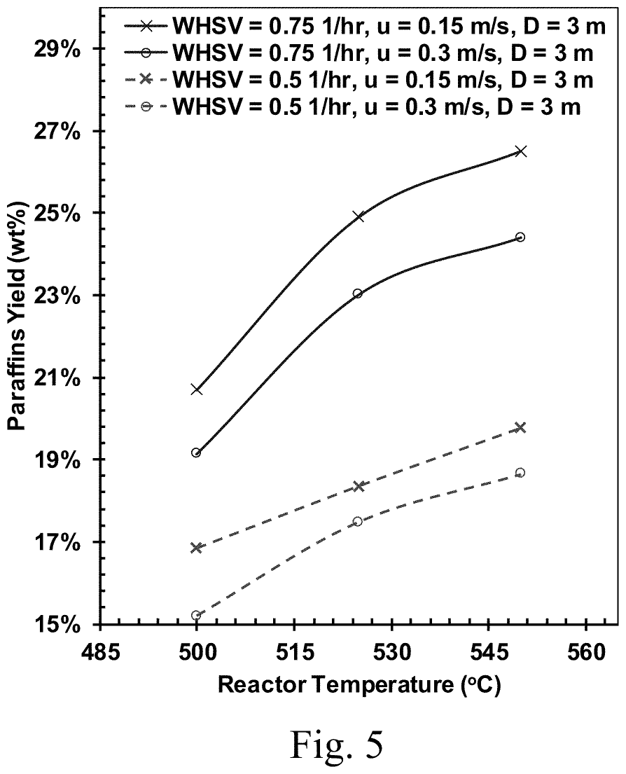

Error! Reference source not found. FIG. 5 shows the impact of temperature on paraffins yield for the four different feed conditions. The data in FIG. 5 Error! Reference source not found. show that higher temperature, higher space velocity, and lower initial superficial gas velocity favor paraffins yield.

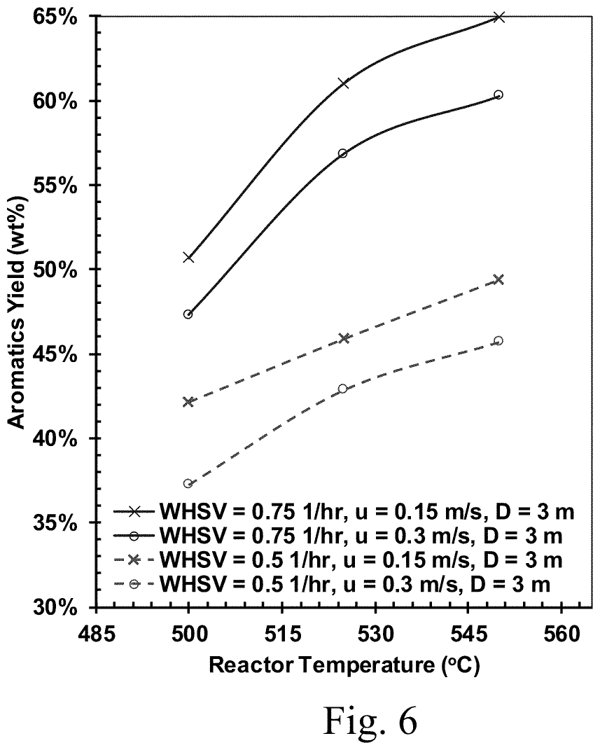

Error! Reference source not found. FIG. 6 shows the impact of temperature on aromatics yield for the four different feed conditions. The data in FIG. 6 Error! Reference source not found. show that higher temperature, higher space velocity, and lower initial superficial gas velocity favor aromatics yield.

### Example 3

TABLE 3 summarizes a comparison of the cylindrical reactor geometry and the conical reactor geometry of the same volume for a catalytic pyrolysis reactor with a nominal capacity of 500 mtpd of feed with a cone angle of 10 degrees and H/D of 4 for the cylindrical geometry.

TABLE 3

| | Cone with same volume |
|---|---|
| Cylindrical at H/D = 4 | D_{cylindrical} = D@~⅓ cone height |

| D (m) | WHSV (1/hr) | D_{bottom} (m) | Dₜₒₚ (m) | H (m) |
|---|---|---|---|---|
| 3.66 | 0.2 | 2.95 | 5.1 | 11.9 |
| 2.69 | 0.5 | 2.19 | 3.71 | 8.7 |
| 2.41 | 0.7 | 1.95 | 3.33 | 7.8 |
| 2.21 | 0.9 | 1.79 | 3.1 | 7.23 |

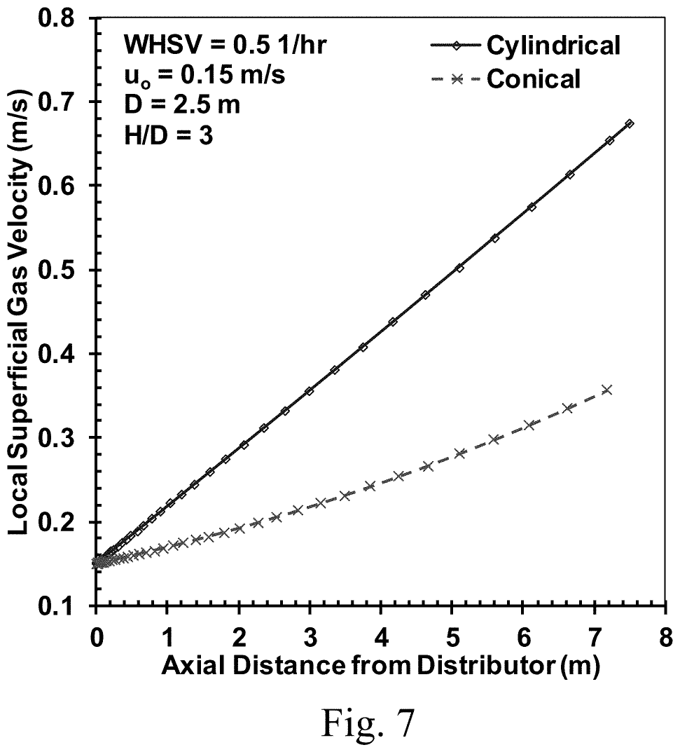

Error! Reference source not found. FIG. 7 shows a comparison of the local superficial gas velocity for a cylindrical reactor and a conical reactor of the same volume for catalytic pyrolysis calculated using a space velocity of 0.5 kg/kg/hr, fluidization gas inlet flow velocity of 0.15 m/sec, and H/D of 4 for the cylindrical reactor and H/D about 4 for the conical reactor where the height of the conical reactor is the height of the conical section and the diameter is the diameter at the bottom of the conical section of the conical reactor. The conical reactor includes an angle of the cone of 10 degrees from the vertical.

The results in FIG. 7 and TABLE 3 show that a conical reactor geometry reduces the increase in superficial velocity of the gases in a fluidized bed along the height of the reactor compared to a cylindrical reactor.

1. A process for converting solid hydrocarbonaceous materials to useful products comprising:
feeding solid hydrocarbonaceous materials and fluidization fluid into a fluidized bed catalytic pyrolysis reactor containing a conversion catalyst,
reacting the hydrocarbonaceous materials to form vapor products and, optionally, solid residue,
withdrawing and recovering the vapor products,
withdrawing catalyst and solids from the catalytic reactor,
feeding the withdrawn catalyst and any solids at or near the bottom of an upflow fluidized bed catalyst regenerator and regenerating catalyst in the regenerator to form hot, regenerated catalyst,
withdrawing the hot, regenerated catalyst from the top of the regenerator,
passing the hot, regenerated catalyst into a fluidized bed catalytic pyrolysis reactor.

2. The process of claim 1 wherein the vapor products are separated from entrained catalyst and solids in one or more cyclones.

3. The process of claim 1 or 2 wherein heat from the catalyst regenerator is used to heat the solid feed materials or fluidization fluid or both.

4. The process of any of the preceding claims wherein an amount of regenerated catalyst is removed and discarded.

5. The process of any of the preceding claims wherein regenerated catalyst is removed and discarded each day in an amount from 0.1 to 5.0, or from 0.5 to 4.0, or from 1.0 to 3.0, or at least 0.1, or at least 0.5, or at least 1, or no more than 5.0, or no more than 4.0 or no more than 3.0% by mass of the separated, regenerated catalyst.

6. The process of any of the preceding claims wherein fresh catalyst is added to the catalytic pyrolysis reactor with the regenerated catalyst or from a separate conduit or a combination of these.

7. The process of any of the preceding claims wherein fresh catalyst is added to the catalytic pyrolysis reactor in an amount of fresh catalyst that is from 0.1 to 5.0, or from 0.5 to 4.0, or from 1.0 to 3.0, or at least 0.1, or at least 0.5, or at least 1, or no more than 5.0, or no more than 4.0 or no more than 3.0% by mass of the regenerated catalyst each day.

8. The process of any of the preceding claims wherein the solid hydrocarbonaceous materials are selected from biomass, polyethylene (PE), polypropylene (PP), polyacetylene, polybutylene, polyolefins, polyethylene terephthalate (PET), polybutylene terephthalate, copolyesters, polyester, polycarbonate, polyurethanes, polyamides, polystyrene (PS), polyacetal, epoxies, polycyanurates, polyacrylics, polyurea, vinyl esters, polyacrylonitrile, polyvinyl alcohol, polyvinylchloride (PVC), poly(methyl methacrylate) (PMMA), polyvinyl acetate, nylon, ethylene-propylene, EPDM, acrylonitrile-butadiene-styrene (ABS), nitrile rubber, natural and synthetic rubber, tires, styrene-butadiene, styrene-acrylonitrile, styrene-isoprene, styrene-maleic anhydride, ethylene-vinyl acetate, nylon 12/6/66, filled polymers, polymer composites, polymer composites comprising natural fibers, plastic alloys, post-consumer recycled polymer materials, municipal solid waste, black liquor, wood waste, or a combination of these.

9. The process of any of the preceding claims wherein the solid hydrocarbonaceous material is selected from among polyethylene, polypropylene, and polystyrene, or mixtures thereof.

10. The process of any of the preceding claims wherein the solid hydrocarbonaceous materials comprise from 30 to 100, or from 40 to 80, or from 45 to 70, or at least 30, or at least 40, or at least 45, or less than 99, or less than 95, or less than 80 percent by mass of a combination of polyethylene (PE), polypropylene (PP), and polystyrene (PS).

11. The process of any of the preceding claims wherein the solid hydrocarbonaceous materials comprise from 1 to 30, or 2 to 20, or 3 to 10, or up to 30, or up to 20 or up to 10, or at least 0.1, or at least 1, or at least 2% by mass biomass.

12. The process of any of the preceding claims wherein the solid hydrocarbonaceous materials comprise from 0.1 to 20, or from 1 to 15, or from 3 to 10, or at least 0.1, or at least 1, or at least 3, or less than 20, or less than 15, or less than 10 percent by mass polyethylene terephthalate (PET).

13. The process of any of the preceding claims wherein the solid hydrocarbonaceous materials comprise from 0.1 to 20, or from 1 to 15, or from 3 to 10, or at least 0.1, or at least 1, or at least 3, or less than 20, or less than 15, or less than 10 percent by mass nylon.

14. The process of any of the preceding claims wherein from 5 to 5000, or from 5 to 2500, or from 100 to 1500, or at least 25, or at least 100 metric tons per day (mtpd) of solid hydrocarbonaceous materials are processed in the catalytic pyrolysis reactor.

15. The process of any of the preceding claims wherein the weight hourly space velocity is from 0.1 to 2.0, or from 0.2 to 1.0, or from 0.25 to 0.75, or at least 0.1, or at least 0.2, or at least 0.25, or less than 2.0, or less than 1.0, or less than 0.75 hr⁻¹.

16. The process of any of the preceding claims wherein the residence time of an average carbon atom within the catalytic pyrolysis reactor is from 0.5 to 180, or from 2 to 100, or from 4 to 80, or from 10 to 60, or from 30 to 60, or at least 5, or at least 10, or at least 30, or less than 180, or less than 100, or less than 80, or less than 60 seconds.

17. The process of any of the preceding claims wherein the catalytic pyrolysis reactor is a fluidized bed reactor that comprises a sparger or distributor, located at or near the bottom of the reactor, that serves to distribute the fluidization fluid.

18. The process of any of the preceding claims wherein the superficial inlet gas velocity of the fluidization fluid entering the reactor is from 0.05 to 1.0, or from 0.1 to 0.7, or from 0.2 to 0.5, or at least 0.1, or at least 0.2, or at least 0.3, or less than 1.0, or less than 0.7, or less than 0.5 meters per second.

19. The process of any of the preceding claims wherein the fluidization fluid is an inert gas, or a hydrocarbon gas, or a recycle stream separated from the products, or a combination thereof.

20. The process of any of the preceding claims wherein the catalytic pyrolysis reactor is from 0.1 to 15, or from 0.3 to 10, or from 3 to 7, or at least 3, or at least 5 meters in diameter.

21-72. (canceled)

73. A process of catalytically pyrolyzing a mixed feed of materials, comprising:
feeding solid hydrocarbonaceous materials and fluidization fluid comprising 0.6 vol % to 10 vol % oxygen into a fluidized bed catalytic pyrolysis reactor containing a conversion catalyst, reacting the hydrocarbonaceous materials to form vapor products and, optionally, solid residue, withdrawing and recovering the vapor products,
withdrawing catalyst and solids from the catalytic reactor,
feeding the spent catalyst and any solids at or near the bottom of an upflow fluidized bed catalyst regenerator,
withdrawing the hot, regenerated catalyst from the top of the regenerator,
separating the hot, regenerated catalyst from the combustion vapors in one or more cyclones, and,
passing at least a portion of the hot, regenerated catalyst into the catalytic reactor.

74-89. (canceled)
